(19) United States
(12) Patent Application Publication
(10) Pub. No.: US 2004/0197914 A1
(43) Pub. Date: Oct. 7, 2004

(54) Viral delivery systems and related manufacture and use

(75) Inventors: David J. Wasilko, Oakdale, CT (US); S. Edward Lee, Waterford, CT (US); William R. Hermans, Millbury, MA (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/700,291

(22) Filed: Nov. 3, 2003

Related U.S. Application Data
(60) Provisional application No. 60/424,238, filed on Nov. 6, 2002.

(51) Int. Cl. C12N 15/86

(52) U.S. Cl. 435/456

(57) ABSTRACT

The invention relates to viral delivery systems and methods for infecting cells with viruses. The viral delivery systems may be cryogenically preserved and they may be used in the manufacture of vaccines, polypeptides, and polynucleotides. The invention includes a cryogenically protected viral delivery system for infecting host cells that comprises a cryogenic vessel and a plurality of virally infected cells in admixture with a cryo-protective agent contained in the cryogenic vessel.

### CROSS REFERENCE TO RELATED APPLICATION

This application claims the benefit of U.S. Provisional Application No. 60/424,238 filed Nov. 6, 2002.

### FIELD OF THE INVENTION

The present invention relates to viral delivery systems and methods for infecting cells with viruses. The viral delivery systems may be cryogenically preserved and they may be used in the manufacture of vaccines, polypeptides, and polynucleotides.

### BACKGROUND OF THE INVENTION

The ability to produce viruses and viral products at high yields and in a consistent (i.e., reproducible) manner is important to numerous industries and medical applications. For example, large quantities of stable viruses are required for use in gene therapy, vaccination protocols, and the production of polypeptides and polynucleotides.

Gene therapy is a method by which recombinant viruses are used to deliver genes encoding therapeutic agents into specific cells of a patient. Gene therapy can be used to treat those diseases that are caused by the inadequate expression of one or more genes that encode for critical proteins, such as, for example, insulin-dependent diabetes, which results from the absence of insulin production in the body, and hemophilia, which results from the inadequate production of Factor VIII or Factor IX in the body. See August, J. T., Ed., Gene Therapy, Advances in Pharmacology, Academic Press Vol. 40 (1997). Targeted gene therapy also uses viruses whose particular genes are deleted and as a result can replicate only in hosts harboring these genes. This permits targeted replication and delivery of the viruses in, for example, either cancer cells or normal cells. See Bradbury, J., The Lancet Oncology, 2:712 (2001); Lindsey, H., The Lancet Oncology, 3:264 (2002). The effective use of gene therapy often requires the quick production of large amounts of virus from a stable and infective virus source.

The effective use of vaccines is also dependent on being able to quickly produce large quantities of virus from a stable and infective virus source. The quick development of vaccines and their abundant availability is critical in combating many human and animal diseases. Delays in producing vaccines and shortfalls in their quantity can cause problems in addressing outbreaks of disease. For example, recent studies suggest that there is cause for concern regarding the long lead times required to produce vaccines against pandemic influenza. See Wood, J. M., Developing Vaccines Against Pandemic Influenza, Philos. Trans. R. Soc. Lond. B. Biol. Sci., 356(1416):1953-60 (2001).

Traditionally, the production of influenza vaccines depended on the availability of embryonated hen eggs for virus growth, which proved to be extremely cumbersome and fraught with numerous disadvantages. See Barrett, O., et al., Development of a Mammalian Cell (Vero) Derived Candidate Influenza Virus Vaccine, Vaccine, 16(9-10):960-68 (1998). Recent technological developments have allowed influenza vaccines to be developed considerably quicker using Vero, BHK/BRS, and MDCK cell lines. See Id.; Merten, O W., Production of Influenza Virus in Cell Cultures for Vaccine Preparation, Adv. Exp. Med. Biol., 397:141-51 (1996). Nonetheless, there is still considerable concern regarding the inability to efficiently produce and distribute influenza vaccines. See Wood, J. M., 356(1416):1953-60 (2001); Morgan, R., Influenza Vaccine. Past and Present, N.J. Med., 98(10):27-34 (2001).

In addition, the industrial scale production of viral polypeptide products, including the production of heterologous proteins using a viral expression system, such as, for example, the ADENOEXPRESS and ADEASY Systems of Q-BIOgene, ADENO-X Adenoviral Expression Systems of Clontech, and the Baculovirus Expression Vector System (“BEVS”), also requires high yields and consistent results. See Miller L. K. Baculovirus As Gene Expression Vectors, Ann. Rev. Microbio., 42: 177-199 (1988). Viral expression systems have become the system of choice in producing viral products, such as, for example, recombinant and non-recombinant polypeptides. In particular, the Baculovirus Expression Vector System has been used extensively in academics and in industry for heterologous protein production. See Summer, M. & Smith, G., A Manual for Baculovirus Vectors and Insect Cell Culture, Tex. Agri. Exp. Stn. Bull., 1555:56 (1987).

Typically, the production of viruses or viral products involves using virus stocks to infect selected host cells. The efficiency of the infection process depends on the relative amount of infectious viral particles used to infect host cells (i.e., the multiplicity of infection or “M.O.I.”). Accordingly, to ensure high yields and consistent production of viral products or viruses, the M.O.I. must be carefully monitored and controlled. In order to determine an appropriate M.O.I., the particular virus stock used must be titered in order to determine the concentration of infectious viral particles.

Typically, the concentration of infectious viral particles in a given virus stock is approximated using a titering assay, such as, for example, the plaque-forming unit assay. The plaque-forming unit assay scores the number of viral plaques formed on a layer of host cells for a given amount of diluted stock, thus giving a titer measured as plaque forming units per milliliter (p.f.u./ml). O'Reilly D. R., Miller, L. K., and Luckow, V. A., Baculovirus Expression Vectors—A laboratory Manual, W.H. Freeman and Company, (1992); Gruenwald S. and Heitz J. H., Baculovirus Expression Vector System: Procedures and Methods Manual, Second Edition, PharMingen, (1993); Gibco BRL Instruction Manual, Guide to Baculovirus Expression Vector System (BEVS) and Insect Cell Culture Techniques, (2002). An alternative to the plaque assay is the tissue culture infective dose procedure (TCID₅₀), which estimates infectivity as a function of intracellular staining for an antigen by direct immunofluorescence. More recently, flow cytometry or FACS (fluorescence-activated cell sorter) assays have been used to measure the number of infected cells in cell cultures. See Morris et al., Virology, 197(1):339-48 (1993). All of these titering procedures require substantial amounts of reagents and laboratory equipment, a plurality of skilled personnel, and can take considerable amounts of time, often from 1 to 2 weeks, to complete.

Most traditional virus stocks are notoriously unstable and must be repetitiously titered prior to every use. Stabilizing the viral particles in a virus stock can be quite difficult. Freezing or lyophilization (freeze drying) has been used to preserve virus stocks, however, both of these techniques often fail or significantly reduce infectivity. For example, LeBlanc & Overstreet report that desiccation for 48 hours inactivated Baculovirus penaei. See Journal of Invertebrate Pathology, 57:277-286 (1991). Loss of virus and/or virus infectivity is so generally a concern for those skilled in the art that the standard method calls for the storage of virus stocks in refrigerators. It is also generally acknowledged by those skilled in the art that virus stocks stored at refrigerated temperatures can lose titer over time. Accordingly, it is strongly recommended in standard laboratory manuals, such as those cited above, to routinely titer the refrigerated virus stocks to ensure consistent infection using such stocks.

As is generally accepted by those of skill in the art, the concentration of infectious viral particles in a given stock will decrease over time. Thus, in order to calculate the appropriate M.O.I. and in order to achieve a concomitant high yield and the consistent production of viral products or viruses, it is strongly recommended and highly desirable that a given virus stock be titered frequently, and more preferably prior to every use.

The repetitious titering of virus stocks greatly multiplies the resources and time necessary to obtain high yields and consistent production of viral products or viruses, a requirement of good manufacturing practices. Minimizing resources and time spent is of critical importance in the commercial industry, where the scale of production is large and cost considerations are of vital importance. For example, the standard method for using the baculovirus expression system calls for the generation of an initial virus stock (often called “P1” for “passage 1”), typically in milliliters, to be followed by several rounds of titering and amplification to arrive at a sizable, typically in liters, working virus stock. See Summer, M. & Smith, G., 1555:56 (1987); Gibco BRL Instruction Manual and Methods to BEVS and Insect Cell Culture Techniques (2002). The standard method also calls for the storage of the large-volume virus stock in a refrigerator. Due to the extreme instability of baculovirus stocks stored in refrigerators, it is not uncommon that rounds of titering, amplification, and expression testing have to be repeated when a virus stock is found reduced or almost depleted in titer upon storage. Accordingly, there remains a need for new, more efficient and time-saving methods for producing viruses and viral products, particularly in a commercial or industrial setting.

### SUMMARY OF THE INVENTION

One embodiment of the present invention provides a cryogenically protected viral delivery system for infecting host cells comprising a cryogenic vessel and a plurality of virally infected cells in admixture with a cryo-protective agent contained in the cryogenic vessel, wherein the concentration of virally infected cells is from 10⁶ cells/ml to 10⁹ cells/ml, wherein the admixture of the virally infected cells and the cryo-protective agent is at a temperature of less than or equal to −20° C., and wherein the viability of cells contained in the cryogenic vessel is at least 50%.

A further embodiment of the present invention provides a method for preparing a cryogenically protected viral delivery system comprising admixing a plurality of virally infected cells with a cryo-protective agent to obtain an admixture having a concentration of virally infected cells of from 10⁶ cells/ml to 10⁹ cells/ml, and freezing at least a portion of the admixture for a time and under conditions sufficient so that the temperature of the frozen admixture is less than or equal to −20° C., and so that the viability of the cells in the frozen admixture is at least 50%.

Another embodiment of the present invention provides a method of virally infecting cells comprising providing a plurality of virally infected cells in admixture with a cryo-protective agent contained in a cryogenic vessel, wherein the concentration of virally infected cells is from 10⁶ cells/ml to 10⁹ cells/ml, wherein the admixture of the virally infected cells and the cryo-protective agent is at a temperature of less than or equal to −20° C., and wherein the viability of cells contained in the cryogenic vessel is at least 50%; providing a plurality of uninfected host cells, wherein the concentration of uninfected host cells is from 10⁵ cells/ml to 10⁷ cells/ml; optionally thawing, washing and/or lysing the virally infected cells; inoculating at least a portion of the uninfected host cells with at least a portion of the virally infected cells; and incubating the inoculated cells in the presence of incubation media for a time and under conditions sufficient to provide a composition in which at least 20% of the cells in the composition are virally infected.

A further embodiment of the present invention provides a method for selecting virally infected cells comprising inoculating a plurality of uninfected host cells with a plurality of viruses; incubating the inoculated cells in the presence of incubation media for a time and under conditions sufficient to obtain a plurality of virally infected cells and monitoring the cell viability and average cell diameter of the incubated cells during the incubation, and stopping the incubation at a point in time when the incubated cells have a viability of at least 50% and an average cell diameter that has increased by at least 0.5 μm.

### BRIEF DESCRIPTION OF THE DRAWINGS

FIG. 1 is a graph illustrating the correlation between extracellular baculovirus concentration and cell size during the incubation of Sf9 insect cells infected with recombinant baculovirus expressing the Beta-site APP (Amyloid Precursor Protein) Cleaving Enzyme (“BACE”). The concentration of extracellular baculovirus is shown on a log scale in p.f.u./ml (-▪ -) and average cell diameter is shown as μm diameter (-● -). Samples of the infected cell culture were removed at various times during incubation (shown in the graph as hours post infection (“hpi”)) and the concentration of extracellular baculovirus and average cell diameter were determined by using a plaque assay and an automated apparatus capable of measuring average cell diameter (the CEDEX apparatus), respectively, as described in Example 1. The average cell diameter peaks at about 18 hpi and plateaues thereafter. The extracellular baculovirus concentration begins to increase at about 12 hpi and peaks at about 40 hpi.

FIG. 2 is a graph illustrating cell viability and viable cell density during the incubation of Sf9 insect cells infected with recombinant baculovirus expressing BACE. The viable cell density is shown on a log scale as number of viable cells/ml (-▪ -) and viability is shown as % viable cells (-● -). Samples of the infected cell culture were removed at various times during incubation (shown in the graph as hpi) and the viable cell density and cell viability were determined by using an automated apparatus capable of measuring viable cell density and cell viability (the CEDEX apparatus), as described in Example 1.

FIG. 3 is a graph illustrating the viability of Sf9 insect cells infected with recombinant baculovirus expressing BACE captured at the 6, 12, 18, and 24 hour time points during the initial incubation (as shown in FIG. 2), after having been separated and collected, admixed with a cryo-protective agent, frozen, thawed at 37° C., and diluted 1:20 in SF 900-II SFM media, as set forth in Example 1. The diluted cells were incubated and samples were removed at various time points (shown in the graph as hpi) and the cell viability was determined by using an automated apparatus capable of measuring cell viability (the CEDEX apparatus), as described in Example 1. The viability is shown as % viable cells for the 6 (-▪ -), 12 (-● -), 18 (-▴ -), and 24 hour (-▾ -) time points.

FIG. 4 is a graph illustrating the increase in average cell diameter of Sf9 insect cells infected with recombinant baculovirus expressing BACE captured at the 6 hour time point during the initial incubation as shown in FIG. 2, after having been separated and collected, admixed with a cryo-protective agent, frozen, thawed at 37° C., and diluted 1:20 in SF 900-II SFM media, as set forth in Example 1. The diluted cells were incubated and samples were removed at various time points (shown in the graph as hpi) and the average cell diameter was determined by using an automated apparatus capable of measuring average cell diameter (the CEDEX apparatus), as described in Example 1. For comparison, the average cell diameter increase of the infected cells during the initial incubation, prior to separating and collecting, is also shown. The average cell diameter is shown as μm diameter for the 6 hour time point (-∘ -) and for the initial incubation prior to collecting (-▪ -). As can be seen when one compares the increase in average cell diameter of the 6 hour sample, which has been separated and collected, admixed with a cryo-protective agent, frozen and thawed, and the initial sample prior to separating and collecting, the increases in cell size closely mimic each other, suggesting that the infection process proceeds as though unabated.

FIG. 5 compares the average cell diameter increase of Sf9 insect cells infected with recombinant baculovirus expressing BACE captured at the 12 hour time point during the initial incubation as shown in FIG. 2, after having been separated and collected, admixed with a cryo-protective agent, frozen, thawed at 37° C., and diluted 1:20 in SF 900-II SFM media, as set forth in Example 1. The diluted cells were incubated and samples were removed at various time points (shown in the graph as hpi) and the average cell diameter was determined by using an automated apparatus capable of measuring average cell diameter (the CEDEX apparatus), as described in Example 1. For comparison, the average cell diameter increase of the infected cells during the initial incubation, prior to separating and collecting, is also shown. The cell size is shown as μm diameter for the 12 hour time point (-Δ -) and for the initial incubation prior to collecting (-▪ -). As can be seen when one compares the increase in cell size of the 12 hour sample, which has been separated and collected, admixed with a cryo-protective agent, frozen and thawed, and the initial sample prior to separating and collecting, the increases in cell size closely mimic each other, suggesting that the infection process proceeds as though unabated.

### DETAILED DESCRIPTION OF THE INVENTION

The present invention provides viral delivery systems and methods of cell infection that may be used in a variety of ways, e.g., in the production of recombinant and nonrecombinant polypeptides, in the production of vaccines, in the production of viral stocks, etc. A principal novel feature of the invention is the preparation, preferably under carefully selected and monitored conditions, of virally infected cells having specified characteristics. Those infected cells are provided in the form of a delivery system, which may be used to infect uninfected cells. The newly infected cells may themselves be used in a variety of ways.

In certain embodiments, the present invention exploits the discovery that virally infected cells can be cryopreserved with little loss of the infective potential of the virus, especially when compared to the unpredictable effects on stability and infectivity that result from cryopreserving viruses outside of a cell. It has been discovered that when virally infected cells are carefully selected, cryopreserved and thawed, the infection cycle resumes upon thawing from the point at which it had been arrested. Because the cryopreservation and thawing have little effect on the stability and viability of the virally infected cells, the viral delivery system and methods of the present invention provide viral products in high yields and with consistent results.

A particularly significant advantage of the present invention is that it renders unnecessary the repetitious and time-consuming titering that is required by conventional methods of producing, storing and using viral stocks. By way of example only, the present invention can reduce the amount of time it takes to produce viral products, including polypeptides and viruses, at an industrial scale using baculovirus from 6-8 weeks to 2-3 weeks.

The cryogenically protected viral delivery system of the present invention comprises a vessel and a plurality of virally infected cells in admixture with a cryo-protective agent contained within the vessel. The plurality of virally infected cells may be obtained by any method desired. However, as the admixture comprising infected cells should have certain properties and characteristics that maximize its performance, the invention provides several useful cell monitoring, selection and storage methods which constitute an additional aspect of the invention.

Viruses

As used herein, the terms “virus” and “viral particle” are used interchangeably and are understood to mean a complete virus, or a portion of a virus that is capable of infecting selected host cells and has the general morphological characteristics of a virus, usually consisting of a genome engulfed by a protein capsid and sometimes a lipid envelope.

Viruses may be characterized by the type of cells they are capable of infecting. Numerous types of viruses can be used and produced in accordance with the present invention. Such viruses include, for example, prokaryotic virus, eukaryotic virus, animal virus, insect virus, avian virus, mammalian virus, primate virus, human virus, murine virus, canine virus, feline virus, bovine virus, porcine virus, equine virus, various vaccine-type virus, or combinations thereof.

Preferred viruses employed in the delivery systems and methods of the present invention include baculovirus, adenovirus, adeno-associated virus, and influenza virus. Also preferred are canarypox virus, infectious bovine rhinotracheitis virus, bovine viral diarrhea virus, parainfluenza 3 virus, bovine respiratory syncytial virus, feline calicivirus, chlamydia virus, canine coronavirus, panleukopenia virus, feline leukemia virus, hepatitis A, hepatitis B, and hepatitis C, human immunodeficiency virus type 1 (HIV-1) and type 2 (HIV-2), cytomegalovirus, human T-lymphotropic virus type I (HTLV-I) and type II (HTLV-II), encephalitis virus, measles virus, mumps virus, rubella virus, polio virus, rabies virus, respiratory syncytial virus, rotavirus, smallpox virus, typhoid vaccine virus, varicella virus, yellow fever vaccine virus, or combinations thereof.

The viruses used in connection with the present invention may be non-recombinant or recombinant. As used herein, the term “recombinant viruses” is understood to mean viruses that contain recombinant polynucleotides. Recombinant polynucleotides are genetically engineered polynucleotides that are typically prepared in vitro by cutting up polynucleotide molecules and splicing together specific polynucleotide fragments, usually from more than one species of organism. Recombinant polynucleotides include polynucleotides from which polynucleotide fragments, such as, for example, genes and portions of genes, have been removed, such as, for example, by homologous recombination or by excising fragments using restriction enzymes and then ligating the ends together. Preferably, the recombinant viruses contain at least one heterologous polynucleotide that encodes a biologically active polypeptide. The heterologous polynucleotides may be integrated into the viral genome or carried on polynucleotide vectors.

Cells

A wide variety of cells may be used in the present invention. Selection of a particular type of cell may be made based upon criteria familiar to those skilled in the art. For example, cells may be chosen based upon their ability to be infected by a given virus and/or their propensity to multiply following infection.

As used herein, the term “host cells” is understood to mean cells that are susceptible to infection with the particular virus of interest (i.e., the virus being used to infect the host cells). Host cells may be eukaryotic or prokaryotic. Preferably, they are eukaryotic. The cells employed in the invention preferably correspond to one of the virus types set forth above, i.e., prokaryotic cells, eukaryotic cells, animal cells, insect cells, avian cells, mammalian cells, primate cells, human cells, murine cells, canine cells, feline cells, bovine cells, porcine cells, equine cells or combinations thereof. When the cells are prokaryotic cells, they are preferably bacterial cells, most preferably E. coli cells.

The host cells may also be, or be derived from, an immortalized cell line. An immortalized cell line is an in vitro cell line that has been transformed to allow unlimited proliferation. Preferably, the immortalized cell line is clonally homologous. A clonally homologous cell line is one in which the cells have identical genetic material. A clonally homologous cell line can be obtained commercially, or by any method commonly used in the art, such as, for example, by using the standard “limited serial dilution cloning” method, utilizing a cell sorting apparatus capable of sorting one cell into one well, such as, for example, Becton, Dickinson and Company's FACS VANTAGE apparatus, or by using standard laboratory “cloning tubes.”

Preferred immortalized cell lines include P6V, Sf9, Sf+(i.e., expresSf+), Sf21, Tn5B1-4 (i.e., High Five), CHO, HEK-293, BHK, NS0, Vero, MDBK, L, L-tk minus, CHO-K1, CHO-dhfr minus, CHO DUKX, NS1, BHK-21, CV-1, U937, HeLa, 293 EBNA, 293 T, 293 F, 293 H, HTB 9, COS-1, COS-7, MDCK, 3T3, 3T3-L1, BALB/3T3, NIH/3T3, 3T3-Swiss albino, WI-38, THP-1, KG-1, HEL 299, MRC-5, MRC-9, WI-38, HepG2, Sp2/0, P3X63Ag8, P3X63Ag8.653, P3X63Ag8U.1, Caco 2, WEHI 3, MCF7, C127, KG-1, IM-9, Daudi, MOP-8, BmN, Bm5, TN368, or combinations thereof. More preferably, the immortalized cell line is CHO, NS0, BHK-21, Sf9, Sf21, High Five, HEK 293, Vero, or combinations thereof. Even more preferably, the immortalized cell line is Sf9, HEK 293, Vero, or combinations thereof.

Making Infected Cells

The delivery system of the present invention requires a plurality of virally infected cells, which can be produced by any method commonly used in the art to infect host cells with virus. A preferred method involves inoculating a plurality of uninfected host cells with a plurality of viruses, incubating the inoculated cells in a composition comprising incubation media for a time and under conditions sufficient to obtain a plurality of virally infected cells, and then separating the plurality of virally infected cells from substantially all spent incubation media and extracellular viral particles and collecting the plurality of virally infected cells in a vessel.

As used herein, the term “uninfected host cells” is understood to mean cells that have not been infected with the particular virus of interest (i.e., the virus being used to infect the uninfected host cells).

Preferably, all of the steps involved in making the virally infected cells (e.g., inoculating, incubating, separating and collecting) are carried out aseptically in order to prevent the presence of contaminating microorganisms. The term “contaminating microorganisms” as used herein is understood to mean any microorganisms other than the host cells and virus that are the primary object of the invention (i.e., the hosts cells and virus used in the inoculation step).

If virus stock is used to inoculate the uninfected host cells, the stock can be either titered or untitered for its initial use. However, after the initial infection, the viral delivery system produced in accordance with the invention can itself be used to inoculate uninfected host cells without any titering.

Titering of virus stock can be accomplished using any method commonly used in the art to titer virus stocks. For example, the plaque forming units (p.f.u.) assay can be used. The p.f.u. assay involves preparing a confluent monolayer of host cells that are susceptible to infection with the stock virus, preparing serial dilutions of the virus in the stock to be titered, plating dilutions of the virus on the monolayer of cells, incubating the cells and virus for a time and under conditions sufficient for the virus to infect the cells and form identifiable plaques, and counting the number of plaques formed on the cell monolayer. The titer of the virus stock can then be mathematically calculated for the undiluted virus stock.

An appropriate amount of the titered virus stock can be determined and used to inoculate the host cells. Determination of the amounts of reagents used, and of the incubation conditions employed, may be made based on factors familiar to those skilled in the art.

Inoculation of the uninfected host cells with virus can be effected using any inoculation procedure commonly used in the art. In the case of infecting cells with human adenovirus, for example, 293-EBNA cells can be grown in 293 SFM II (Gibco # 11686) media supplemented with 5% fetal bovine serum to a final cell density of about 0.75×10⁶ viable cells/ml, and infected with a small sample, such as, for example, 0.01-1 ml, of a virus stock. Preferably, uninfected host cells are grown in shake flasks containing incubation media, such as, for example, in the case of baculovirus, Sf9 cells can be grown in SF-900-II-SFM (Gibco #10902-096) media containing FBS, to a final cell density of about 0.75×10⁶ viable Sf9 cells/ml. The cells are then infected with a small sample, such as, for example, 0.1-20 ml, of a virus stock. Preferably, the virus stock used has a titer of at least 1×10⁷-1×10¹¹ viral particles/ml. Preferably, enough virus stock is used to insure that the cells are inoculated with virus at an appropriate multiplicity of infection (“M.O.I.”). One of skill in the art will recognize that the appropriate M.O.I. will vary depending on the type of virus used. For example, when using baculovirus, the appropriate M.O.I. is preferably at least 0.01, more preferably at least 0.1, most preferably at least 1. When using adenovirus, the appropriate M.O.I. is preferably at least 1, more preferably at least 10, most preferably at least 50.

Once the uninfected host cells have been inoculated, the inoculated cells should be incubated for a time and under conditions sufficient to obtain a plurality of virally infected cells. The inoculated cells can be incubated under conditions commonly used in the art for incubating virally inoculated cells, such as, for example, by shaking the inoculated cells in a shake flask at a proper incubation temperature. One of skill in the art will recognize that for different viruses and host cell types, the incubation conditions, including the proper incubation temperature, will vary. Preferably, the incubated cells are incubated in a shake flask while gently shaking at 27-37° C.

Preferably, the infection and incubation are conducted under conditions sufficient to insure that a large percentage of the total incubated cells in the vessel are virally infected. The percentage of cells that are infected is particularly affected by the amount of virus used to infect the cells and the efficiency of the infection process. The percentage of virally infected cells can be determined by any method commonly used in the art for this purpose.

Preferably, the GFP method, which utilizes a recombinant version of the virus of interest that expresses Green Fluorescence Protein (“GFP”), is used to determine the percentage of virally infected cells. The GFP method involves using the GFP-recombinant virus to infect host cells. The cells that are infected with the GFP-recombinant virus emit green light (i.e., fluorescence). A sample of the cells is then removed, serially diluted, and visually analyzed under a fluorescence microscope, such as, for example, a Nikon TE 300 microscope with an FITC cube (catalog No. R620-07). The number of green light emitting cells and the total number of cells are counted, and the numbers computed to give a percentage of virally infected cells.

More preferably, the percentage of virally infected cells is determined using flow cytometry. Flow cytometry has become a very useful method for the automated, rapid analysis of cells in suspension at the single-cell level. See Jensen and Horan, P. K., Flow Cytometry: Rapid Isolation and Analysis of Single Cells, Methods Enzymol., 171:549-581 (1989). Flow cytometry has been used extensively in virological research. See McSharry, J. J., Use of Flow Cytometry in Virology, Clin. Microbiol. Rev., 7:576-604 (1994). Most applications of flow cytometry in virological research are based on virus detection by immunofluorescence, such as, for example, the detection of herpes simplex virus (McSharry et al., J. Clin. Microbiol., 28:1864-1866 (1990)), human immunodeficiency virus (Yang et al., Cytometry, 21:197-202 (1995)), rotavirus (Barardi et al., J. Virol. Methods, 74:31-38 (1998)), and rabies virus (Bordignon et al., J. Virol. Methods, 105:181-186 (2002)).

The flow cytometry method involves detaching host cells infected with the virus of interest from the surface on which they have grown (only if necessary, such as, for example, if the virally infected cells are incubated in Petri dishes instead of in suspension culture). The cells are then washed, using PBS containing TWEEN 20, and permeated by treatment with a solution containing p-formaldehyde and lysis reagent, such as, for example, Becton Dickinson No. 349202. The washed and permeated cells are then incubated with fluorescin conjugated antibody that is specific for a protein expressed by the virus of interest, such as, for example, a virus nucleocapsid protein. The antibody treated cells are then analyzed using flow cytometry, preferably by using a Fluorescence Activated Cell Sorter (“FACS”), such as, for example, the Becton Dickinson FACS VANTAGE apparatus. One of skill in the art will readily recognize that antibodies that are coupled to a fluorescent agent and that are specific for proteins expressed by viruses of interest are well known and can readily be obtained for use with flow cytometry.

Preferably, the uninfected host cells are inoculated and the inoculated cells are incubated for a time and under conditions sufficient to provide that the virally infected cells represent at least 20% of the total number of incubated cells, more preferably at least 40%, even more preferably at least 60%, most preferably at least 80%.

Cell Viability and Average Cell Diameter

As indicated above, the cells in the delivery system of the invention may be produced, selected and collected so that they have certain performance characteristics and properties. Already mentioned is the percentage of cells that are virally infected. Two other characteristics and properties of the collected cells are cell viability and average cell diameter.

As used herein “cell viability” is understood to mean the percentage of cells that are viable as determined using the Trypan Blue Exclusion Method as described in Doyle, A., Griffiths, J. B., and Newell, D. G., Cell & Tissue Culture: Laboratory Procedures, Module 4B:1—Hemocytometer Cell Counts and Viability Studies, 4B: 1.1-4B: 1.5, John Wiley & Sons, West Sussex, England (1994). The Trypan Blue Exclusion Method may be carried out manually or with the aid of an automated device, such as those described and exemplified herein.

One of skill in the art will readily recognize that average cell diameter can be directly measured, or can be calculated using some other cell measurement, such as, for example, the cell circumference, the cell radius or the cell volume. Once again, automated devices such as those described and exemplified herein may be used. One of skill in the art will also recognize that not all cells are exactly round and that cell shapes might vary slightly, thus diameter measurements for individual cells will vary accordingly.

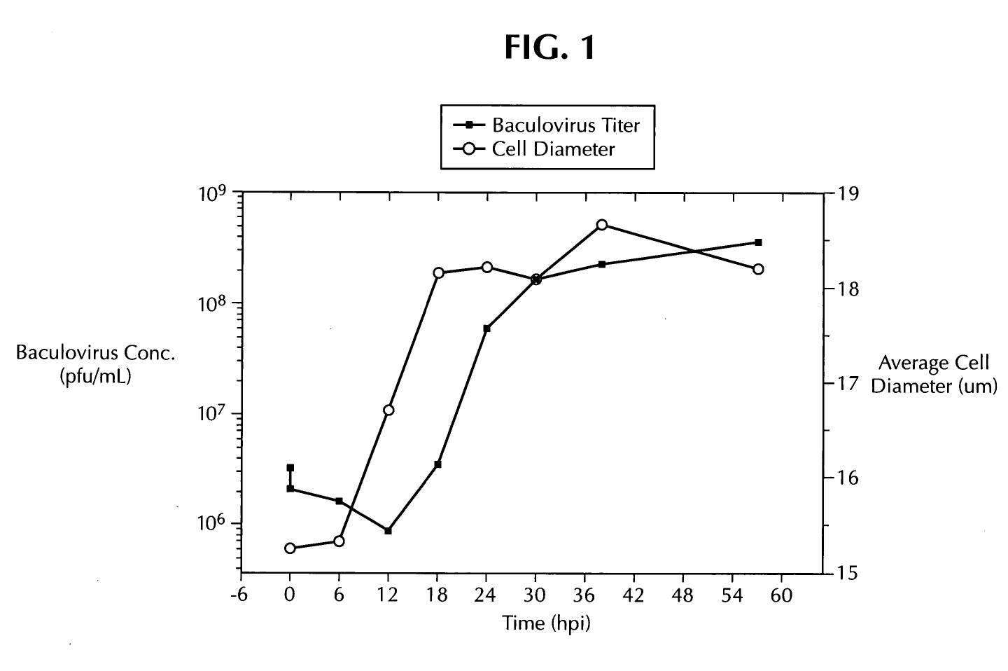

Cell viability and the average cell diameter of a population of host cells inoculated with virus will generally vary as the infectious process proceeds during incubation. Typically, the infected cells swell (i.e., increase in average cell diameter) until they reach a maximum average cell diameter and then the average cell diameter plateaus and remains essentially constant for a period of time. FIG. 1 illustrates and Example 1 describes the increase in average cell diameter observed during the incubation of Sf9 insect cells infected with recombinant baculovirus expressing BACE. As can be seen in FIG. 1, the average cell diameter of the cells increases until about 18 hours post infection (“hpi”) and then plateaus until the last time point at 56 hpi.

It is believed that the increase in average cell diameter is due to virus replication occurring inside of the infected cells and, consequently, the cells filling up with virus. One of skill in the art will recognize that the increase in average cell diameter of the infected cells will vary depending on the type of virus and host cells used.

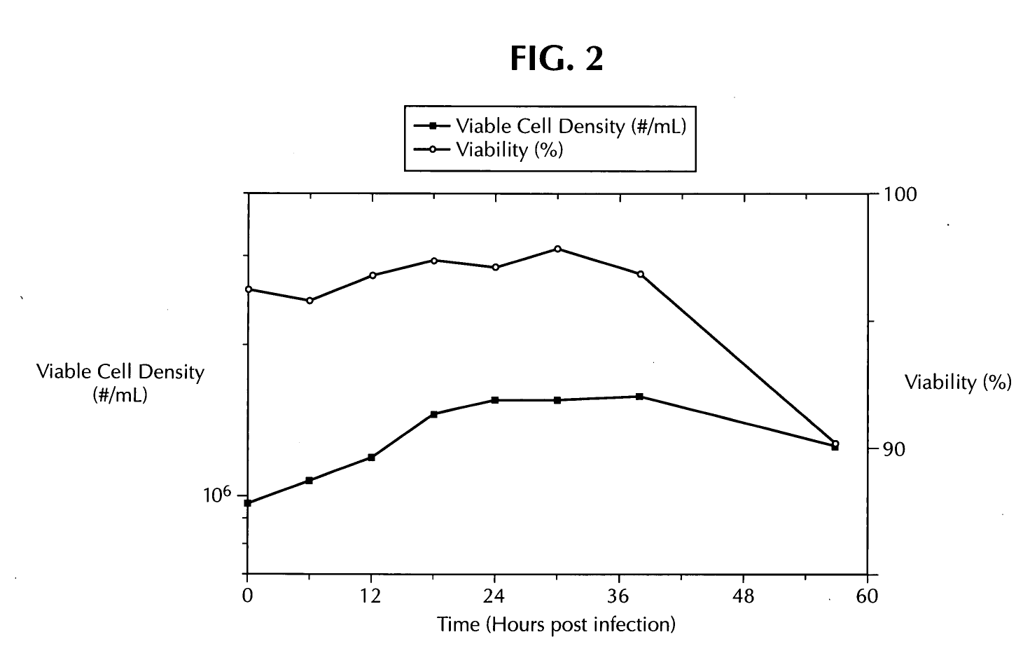

At some point during the incubation of virally infected cells, the viability of the cells begins to decrease. FIG. 2 illustrates and Example 1 describes the decrease in cell viability observed during the incubation of Sf9 insect cells infected with recombinant baculovirus expressing BACE. FIG. 2 illustrates that the viability of the cells begins to decrease at about 30 hpi. A comparison of FIG. 2 with the average cell diameter graph in FIG. 1 illustrates that the cell viability decreases at a point in time after the average cell diameter has plateaued.

It is believed that the viability decreases in part due to the virally infected cells “bursting” and releasing virus. This theory is supported by two observations: the first is that, as illustrated in FIG. 1 and described in Example 1, the amount of extracellular virus found in solution during the incubation begins to increase at about the same time as the average cell diameter increases, and peaks a short time after the average cell diameter begins to plateau; the second is that if one compares FIGS. 1 and 2, it is apparent that the increase in extracellular virus correlates with the decrease in viability. Thus, it is postulated that once the cells are full of virus and reach their maximum average cell diameter they begin to burst and release virus, at which point the cell viability decreases and the amount of extracellular virus present in solution increases.

Accordingly, based on the observations set forth above, it is preferred that the virally infected cells are separated and collected at a point during the incubation shortly after the average cell diameter has plateaued, thereby insuring that the virally infected cells are full of virus, and when the cell viability is high and the amount of extracellular virus is low, thereby insuring that viruses are collected while still inside the cells.

Monitoring

It is generally preferred that cell viability and average cell diameter are monitored during the incubation of the inoculated cells. Typically, monitoring involves removing a sample of the incubation culture during the incubation process and determining the cell viability and average cell diameter of at least a portion of the cells in the sample. Preferably, the procedure used to make the determinations is relatively quick.

The reason why a relatively quick procedure is preferred is because the virally infected cells should be separated and collected when the viability and average cell diameter of the incubating cells reach the desired values. If the procedures used to measure viability and average cell diameter take too long, the viability and average diameter of the cells might change during the time that it takes to carry out the procedures, particularly if the incubation proceeds during that time. Thus, if the procedure takes too long, by the time it is determined that the cells have reached the appropriate viability and average cell diameter, the viability and average cell diameter of the cells might have changed substantially.

Preferably, viability and/or average cell diameter is determined by using an automated apparatus that is capable of measuring/monitoring cell size, generally by average cell diameter, and cell viability. Suitable devices for this purpose include the VI-CELL apparatus manufactured by Beckman, which is an automated system built on an imaging flow cell for determining cell viability using the standard Trypan Blue staining protocol, and cell size by digital image processing; the PERSONAL CELL ANALYZER manufactured by Guava, which determines cell size through light scattering technology and cell viability based on differential permeability of DNA-binding dyes in the VIACOUNT reagent (Guava #4000-0040); and the CEDEX apparatus, manufactured by Innovatis.

The CEDEX apparatus determines average cell diameter and cell viability using a standardized and fully automated procedure based on the Trypan Blue Exclusion Method. The Trypan Blue Exclusion Method is based on the principle that dying or dead cells attract dye and stain while viable cells repel the dye and do not stain. See Doyle and Newell. The CEDEX apparatus automates the basic manual steps of the Trypan Blue Exclusion Method, thus greatly decreasing the amount of time required to carry out the procedure. Generally, the cell viability results generated by the CEDEX apparatus and those obtained using the manual Trypan Blue Exclusion Method are statistically the same. See Kwok, S. M. et al., Evaluation of an Automated Cell Density Examination (CEDEX) System, Paper BIOT-229, 224 ACS National Meeting, Boston Mass., August 18-22 (2002).

The basic CEDEX procedure involves the aspiration of Trypan Blue dye to a sample cup containing the cell suspension to be tested. Preferably, a 0.2% Trypan Blue solution is used. Preferably, the cell suspension has a cell density range from 5×10⁴-5×10⁷ cells/ml. The cell suspension/dye admixture is then mixed by pump action between the syringe and the sample cup and then incubated for an exact amount of time that is used for each of the numerous automated measurements. The stained cell suspension is then transported in small amounts to a measurement chamber wherein a set number of microscopic images of the sample are taken and transferred to the computer analysis software via an integrated interface in the measurement system. The CEDEX apparatus then provides measurements of average cell diameter and cell viability.

Monitoring cell viability and average cell diameter typically involves removing a small aliquot, such as, for example, 0.1-100 ml, of the incubation culture at various time points during the incubation, optionally diluting the sample, and determining the cell viability and average cell diameter of the cells in the aliquot. Determining the cell viability and average cell diameter of the cells in the aliquot typically involves placing at least a portion of the cells in an automated apparatus capable of measuring cell viability and average cell diameter. Preferably, one of the automated apparatuses set forth above for measuring cell viability and average cell diameter is used, such as, for example, the CEDEX apparatus and the basic CEDEX procedure set forth above. Preferably, determining the cell viability and average cell diameter of the cells in the aliquot can be carried out in less than 30 minutes, more preferably in less than 10 minutes, most preferably in less than 5 minutes, following removal of the aliquot from the incubation culture.

Typically, an initial average cell diameter determination is made within 5 minutes following the admixing of virus stock with an uninfected cell population. The initial average cell diameter determination represents the average cell diameter of uninfected cells of the same type. The initial average cell diameter determination is preferably determined shortly after the admixing of virus stock with an uninfected cell population in order to insure that the initial determination is made under the same media conditions as later determinations of average cell diameter. The term “same media conditions” as used herein, is understood to mean that the media in which the cells are in solution has the same or very similar ingredients, particularly with regards to the osmolarity of the media, which can noticeably affect cell size. One of skill in the art will recognize that as the cells are incubated in the media, the cells will generally grow and metabolize, therefore changing the contents of the media, particularly with regards to the concentration of nutrients. Accordingly, the media conditions for the initial determination of average cell diameter will likely not be exactly the same as the media conditions for determinations made later in the incubation process.

As the incubation proceeds, the average cell diameter will increase to a maximal plateau value. In accordance with various embodiments of the delivery system of the invention, the incubated cells are preferably separated and collected at a point in time when the average cell diameter of the incubated cells has increased by at least 0.5 μm, as compared to the initial average cell diameter determination, more preferably by at least 1 μm.

Further in accordance with various embodiments of the delivery system of the invention, the incubated cells are preferably separated and collected at a point in time when the viability of the incubated cells is above 50%, more preferably above 70%, most preferably above 90%.

A further embodiment of the present invention is a method for selecting virally infected cells that involves monitoring the viability and average cell diameter during the incubation of host cells that have been inoculated with a virus, and stopping the incubation at a point in time when the incubated cells have a viability of above 50%, more preferably above 70%, most preferably above 90%, and an average cell diameter that has increased by at least 0.5 μm, as compared to the initial average cell diameter determination, more preferably by at least 1 μm.

Still another embodiment involves stopping the incubation at a point in time when the incubated cells have a viability of above 50% and an average cell diameter that has increased by at least 0.5 μm, more preferably when the cells have a viability of above 50% and an average cell diameter that has increased by at least 1 μm, even more preferably when the cells have a viability of above 90% and an average cell diameter that has increased by at least 0.5 μm, most preferably when the cells have a viability of above 90% and an average cell diameter that has increased by at least 1 μm.

Monitoring cell viability and average cell diameter can be carried out using any procedure commonly used in the art. Preferably, monitoring is carried out using an automated apparatus, such as, for example, the VI-CELL apparatus manufactured by Beckman, the PERSONAL CELL ANALYZER manufactured by Guava, or the CEDEX apparatus, manufactured by Innovatis. Most preferably, monitoring is carried out using the CEDEX apparatus.

The phrase “stopping the incubation” as used herein shall be understood to mean arresting cell and viral activities, such as, for example, growth and replication. A person of skill in the art will readily recognize that there are many methods for arresting cell and viral activities, such as, for example, by lowering the temperature of the cells and/or viruses, thus slowing down the rate of cellular and viral activities, and/or by separating the cells and/or viruses from nutrients. One of skill in the art will also recognize that the method used to arrest cell and viral activities likely will not completely stop these activities.

The incubation can be stopped by any method commonly used in the art for stopping an incubation, such as, for example, by pelleting the cells in the incubation mixture via centrifugation and decanting the supernatant, thus separating the cells from nutrients present in the incubation media. Preferably, centrifugation involves placing 50 ml portions of the incubation mixture in 50-ml conical centrifuge tubes and centrifuging at 150×g for 10 minutes. Alternatively the incubation can be stopped by lowering the temperature of the incubated cells, such as, for example, by placing the incubation mixture on ice. Preferably, the temperature of the cells in the incubation mixture is lowered to at least 10° C. or lower.

Typically, the incubation is stopped shortly after the determinations made during the monitoring step indicate that the incubated cells have achieved the desired cell viability and average cell diameter. Preferably, the incubation is stopped within 12 hours from the time that the determinations indicate the desired values, more preferably within 2 hours, even more preferably within 30 minutes, still more preferably within 10 minutes, most preferably within 5 minutes.

Separation/Isolation

Once the incubation has proceeded for a time and under conditions sufficient to obtain a plurality of virally infected cells, and the incubated cells have one or more of the characteristics described above, the virally infected cells are separated and collected, which will typically stop the incubation process.

Separating and collecting the plurality of virally infected cells in a vessel can be effected by any method, or combination of methods, commonly used in the art for this purpose. A person of skill in the art will recognize that the separating and collecting steps can be performed simultaneously or separately, and if performed separately, the two steps can be performed in any order. Preferably, the virally infected cells are separated and collected simultaneously by centrifugation and by decanting the supernatant.

Centrifugation involves placing the incubated culture, including the virally infected cells and spent incubation media, in a vessel and centrifuging at, for example, about 150×g for about 5-10 minutes. An example of a centrifuge apparatus is the CENTRA CL2 manufactured by IEC International Equipment Company. Once the cells have been pelleted, the supernatant, which contains spent incubation media and exrtracellular viral particles, can be removed, preferably by decanting or pipetting, or by decanting the majority of the spent incubation media and pipetting off as much of the remaining spent incubation media as possible.

Preferably, the virally infected cells are collected in a vessel having a size of at least 10 ml, more preferably at least 50 ml, even more preferably at least 250 ml, most preferably at least 1000 ml. Any vessel commonly used in the art to collect cells can be used. Preferably, the vessel is a 15 ml or 50 ml conical tube (Falcon/Corning), or a 250-ml, 500-ml, or 1000-ml centrifuge bottle (Nalgene).

Preferably, the virally infected cells are collected and separated in a manner such that the virally infected cells contained in the vessel are substantially free of spent incubation media and substantially free of extracellular viral particles.

Spent incubation media is understood to mean incubation media in which cells have been grown. As used herein, “substantially free of spent incubation media” means that the spent incubation media represents less than 50% of the total volume of the collected cells. More preferably, the amount of spent incubation media remaining after the separating and collecting steps is less than 30%, most preferably less than 10%.

Accordingly, the small amount of spent incubation media that would remain after the supernatant is decanted, pipetted, or aspirated from a centrifuged culture of incubated cells would not be excluded from the meaning of “substantially free of spent incubation media” set forth above.

The actual amount of spent incubation media remaining after the separating and collecting steps can be determined by measuring the amount of a “marker” substance that is present in the spent incubation media. For example, if the spent incubation media contains glucose, the concentration of glucose in the undiluted spent incubation media that was removed is determined (e.g., the supernatant in the centrifugation protocol set forth above). The pelleted cells are then resuspended in some volume of solution that does not contain glucose and which does not disrupt the integrity of the cells, the cells are then collected and separated again and the glucose concentration of the removed solution is determined. The glucose concentration of the removed solution should represent that contributed by the small amount of spent incubation media that remained after the initial collecting and separating steps. Accordingly, using this information, one can mathematically calculate the amount of spent incubation media that remained after the initial separating and collecting steps. The concentration of glucose can preferably be determined by a biochemical analyzer, such as, for example, the YSI 2700 SELECT manufactured by YSI Incorporated.

Extracellular viral particles are those viruses and portions of viruses that are located outside of the confines of the wall or cytoplasmic membrane of a cell. As used herein, “substantially free of extracellular viral particles” shall be understood to mean that the extracellular viral particles are less than 10% of the total number of viral particles present. More preferably, the extracellular viral particles present after the separating and collecting steps is less than 3% of the total number of viral particles present, most preferably less than 1%.

In order to calculate the percentage of the total number of viral particles that are extracellular viral particles, one typically determines the total number of viral particles present and the number of extracellular viral particles present and then mathematically calculates the percentage.

The total number of viral particles present after the separating and collecting steps can preferably be determined by diluting the cells and remaining spent incubation media to a final cell concentration of 10⁶ cells/ml, lysing the cells, and performing a p.f.u. assay on the lysate.

The number of extracellular particles present after the separating and collecting steps can preferably be determined by resuspending the cells in liquid media, such as, for example, the same media used to incubate the cells, gently removing the cells from the liquid media, such as, for example, by centrifugation or by filtering through a filter with a small enough pore size to allow the media and extracellular viral particles to pass, but not the cells, and then performing a p.f.u. assays on the separated media.

Preferably, virally infected cells represent at least 20% of the total number of separated and collected cells in the vessel, more preferably at least 40%, even more preferably at least 60%, most preferably at least 80%.

The percentage of virally infected cells in the vessel can be determined by any method commonly used in the art. Preferably, the method used to determine the percentage of virally infected cells utilizes a GFP-recombinant virus, such as, for example, the preferred GFP method set forth above for measuring the percentage of virally infected cells in the incubation process. More preferably, the percentage of virally infected cells is determined using flow cytometry, such as, for example, the preferred flow cytometry method set forth above for measuring the percentage of virally infected cells in the incubation process.

Preferably, the separated and collected cells have a viability of at least 50%, more preferably at least 70%, most preferably at least 90%.

Preferably, the average cell diameter of the separated and collected cells is at least 0.5 μm greater than the average cell diameter of uninfected cells of the same type, more preferably 1 μm greater.

Preferably, viability and/or average cell diameter are determined using one of the automated apparatuses set forth above for measuring cell viability and average cell diameter, such as, for example, the VI-CELL apparatus manufactured by Beckman, the PERSONAL CELL ANALYZER manufactured by Guava, or the CEDEX apparatus, manufactured by Innovatis. Most preferably, viability and/or average cell diameter are determined using the CEDEX apparatus. Typically, a sample of the cells is removed, serially diluted, and analyzed using the CEDEX apparatus.

Preferably, the separated and collected virally infected cells are kept on ice, and treated delicately, such as, for example, by not overly agitating the cells or forming bubbles with the culture, during the collecting and separating steps. Such precautions should help preserve the integrity of the cellular membranes and concomitantly help maintain cell viability, average cell diameter and the percentage of the total number of cells in the vessel that are virally infected cells, virtually unchanged during the collecting and separating procedure. Accordingly, these values should be nearly the same as those values measured during incubation, prior to collecting and separating the virally infected cells.

Preparing the Cryopreserved Delivery Systems

In a preferred embodiment of the present invention, the cryogenically preserved viral delivery system is prepared by selecting a plurality of virally infected cells having one or more of the desirable characteristics described above, admixing the virally infected cells with a cryo-protective agent, and then carefully freezing the cells to arrest the infective cycle of the viruses within the cells. The freezing protocols should be selected to preserve the viability of the cells and so that the viruses in the cells may resume their infective cycle upon thawing.

Preferably, all of the steps involved in making the cryogenically protected viral delivery system (e.g., admixing, aliquoting, freezing) are carried out aseptically in order to prevent the presence of contaminating microorganisms. The term “contaminating microorganisms” as used herein is understood to mean any microorganisms other than the host cells and virus that are the primary object of the invention (i.e., the host cells and virus used in the inoculation step).

In general, the cryogenically protected viral delivery system of the present invention includes a cryogenic vessel containing an admixture of a plurality of virally infected cells and at least one cryo-protective agent.

The cryo-protective agent can be any substance, or combination of substances, commonly used in the art to protect cells from the adverse effects of the freezing process. Preferred cryo-protective agents are DMSO, glycerol, serum, serum albumin, skim milk, dextran, polyvinylpyrrolidone, sucrose, trehalose and mixtures thereof. More preferably, the cryo-protective agent is DMSO, serum, serum albumin, glycerol or mixtures thereof. As used herein “serum” is defined as the fluid that is left over when blood clots. A preferred serum is fetal bovine serum. Preferably, the concentration of cryo-protective agent present in the composition is from about 0.1% (v/v, or w/v if in solid form) to about 90% (v/v, or w/v if in solid form), most preferably from about 1% (v/v, or w/v if in solid form) to about 20% (v/v, or w/v if in solid form). One of skill in the art will recognize that the optimum concentration of cryo-protective agent will vary depending on the specific type of cryo-protective agent used.

A variety of vessels, including cryogenic vessels, may be used to contain the virally infected cells and the cryo-protective agent. The size and composition of the cryogenic vessel may be chosen as needed.

Preferred cryogenic vessels include polypropylene test tubes, cryotubes, vials or bottles. More preferably the cryogenic vessels are one of the following vessel types (all catalog numbers refer to the VWR catalog): VWR-brand low-temperature freezer vials, polypropylene, sterile, with cap, in the following sizes: 1 ml, cat. no. 66008-251; 2 ml, cat. no. 66008-284; and 5 ml, cat. no. 66008-400, Corning Costar-brand BIOFREEZE vials, polypropylene, sterile, with screw cap, in the following sizes: 1.2 ml, cat. no. 29442-536; 2.0 ml, cat. no. 29442-540; 2.0 ml, cat. no. 29442-538; and 6.0 ml, cat. no. 29442-542, Corning-brand cryogenic vials, polypropylene, sterile, with closures, in the following sizes: 1.2 ml, cat. no. 66021-972; 2.0 ml, cat. no. 66021-976; 2.0 ml, cat. no. 66021-974; 4.0 ml, cat. no. 66021-978; 5.0 ml, cat. no. 66021-980; 1.2 ml, cat. no. 66021-940; 2.0 ml, cat. no. 66021-942; 4.0 ml, cat. no. 66021-946; 4.0 ml, cat. no. 66021-948; 5.0 ml, cat. no. 66021-950; 5.0 ml, cat. no. 66021-970; 1.0 ml, cat. no. 66021-919; and 2.0 ml, cat. no. 66021-920, Nalgene-brand cryogenic vials, polypropylene, sterile, with screw cap, in the following sizes: 1.2 ml, cat. no. 66008-706; 2.0 ml, cat. no. 66008-728; and 5.0 ml, cat. no. 66008-732, Wheaton-brand CRYULE vials, polypropylene, sterile, with screw cap, in the following sizes: 1 ml, cat. no. 66008-874; and 2 ml, cat. no. 66021-750, Wheaton-brand CRYULE vials, polypropylene, sterile, with screw stopper, in the following sizes: 2 ml, cat. no. 66008-952; 4 ml, cat. no. 66008-953; 5 ml, cat. no. 66008-954; 1.2 ml, cat. no. 66008-955; 2 ml, cat. no. 66008-956; and 4 ml, cat. no. 66008-957, Nunc brand CRYOTUBE vials, polypropylene, sterile, with screw stopper, in the following sizes: 1.0 ml, cat. no. 66021-993; 1.8 ml, cat. no. 66021-986; 3.6 ml, cat. no. 66021-989; and 4.5 ml, cat. no. 66021-991, Nalgene-brand square media bottle, PETG, sterile, in the following sizes: 30 ml, cat. no. 16159-126; 60 ml, cat. no. 16159-128; 125 ml, cat. no. 16159-130; 250 ml, cat. no. 16159-132; 500 ml, cat. no. 16159-134; 1000 ml, cat. no. 16159-136; and 2000 ml, cat. no. 16159-138. Particularly preferred vessels are the 1.2-ml, 2.0-ml, and 5.0-ml low-temperature freezer vials of the VWR brand (VWR #66009-251, 66008-284, and 66008-400).

Preferably the volume of the cryogenic vessel is less than or equal to 250 ml, more preferably less than or equal to 30 ml, most preferably less than or equal to 6 ml. Preferred cryogenic vessels are vials having a volume of less than or equal to 12 ml, more preferably vials having a volume of less than or equal to 6 ml. Most preferably, the vessels are polypropylene vials having a volume of less than or equal to 6 ml.

Any desirable method may be employed to bring the infected cells into admixture with the cryo-protective agent inside the cryogenic vessel.

When the plurality of virally infected cells is provided by one of the protocols described above, for example, it may be in the form of a pellet of centrifuged cells. The cells are preferably substantially free of extracellular virus particles and substantially free of spent incubation media. The pellet may be optionally washed, admixed with the cryo-protective agent and aliquotted into a plurality of cryogenic vessels. Alternatively, the infected cells may be aliquotted and admixed with the cryo-protective agent in the cryogenic vessel.

The virally infected cells and the cryo-protective agent may be admixed in any manner commonly used in the art. Preferably, the cryo-protective agent(s) is added to the cells using a pipette. More preferably the cryo-protective agent is added as part of a freezing medium which, in addition to one or more cryo-protective agents, also contains a buffer or an incubation media, such as, for example SF-900 II SFM. The cells are preferably suspended in this freezing medium by gentle shaking or vortexing.

Preferably, the virally infected cells are admixed with the cryo-protective agent to provide that the concentration of the virally infected cells is from 10⁶ cells/ml to 10⁹ cells/ml, more preferably from 5×10⁶ cells/ml to 5×10⁸ cells/ml. The concentration of virally infected cells can be determined by any method commonly used in the art, such as, for example, by removing a small sample of the cells, serially diluting the sample, placing a small amount of the serial dilutions on a slide, using a microscope to count the number of cells in a given volume of the diluted sample, and then mathematically calculating the concentration of total cells in the undiluted admixture. With this information and the percentage of virally infected cells as determined by the GFP or flow cytometry methods set forth above, one can calculate the concentration of virally infected cells in the vessel.

Preferably, once the virally infected cells and the cryo-protective agent have been admixed and aliquoted into the cryogenic vessels, each of the cryogenic vessels contain from 10⁵ to 10¹² virally infected cells, more preferably from 10⁶ to 10¹⁰ virally infected cells, most preferably from 5×10⁶ to 5×10⁸ virally infected cells. Preferably, the volume of the admixture contained in each of the cryogenic vessels is from 0.1 ml to 200 ml, more preferably from 0.5 ml to 20 ml, most preferably from 1 ml to 4 ml. This is readily done by selecting an appropriate volume of cryo-protective agent to be admixed with a plurality of virally infected cells of known volume and concentration, and by aliquoting known amounts into the cryogenic vessels. Preferably, the volume of the admixture contained in the cryogenic vessel represents at least 10% of the total volume of the cryogenic vessel, more preferably at least 40%, even more preferably at least 66%%, most preferably at least 80%.

Freezing

The admixture of virally infected cells and cryo-protective agent is preferably frozen under controlled conditions in a cryogenic vessel. Any suitable freezing protocol may be used, keeping in mind that the conditions should be selected so that the viability and infectivity of the cells are preserved as best as possible. As used herein, the term “frozen” is understood to mean in a solid form due to the abstraction of heat.

Preferably, the admixture is frozen under conditions to provide that the cell viability, average cell diameter and the percentage of the total number of cells in the vessel that are virally infected cells remain virtually unchanged from those values measured during incubation. Accordingly, the preferred ranges for these measurements remain unchanged from the preferred values cited above. However, inasmuch as the freezing process involves complex phenomena that can affect cell stability, it is acceptable that the frozen cells retain significantly all of the desirable characteristics described above. For example, allowance may be made for the loss of up to 50% of the desired (pre-freezing) cell viability.

Accordingly, it is important that the virally infected cells undergo a controlled cooling process that controls the size and formation of ice crystals and hence preserves the stability of the cells. Such precautions should help preserve the integrity of the cellular membranes and concomitantly help maintain the cell viability, average cell diameter and the percentage of the total number of cells in the vessel that are virally infected cells, unchanged during the freezing procedure. The cell viability, average cell diameter and the percentage of the total number of cells in the vessel that are virally infected cells can be determined for the virally infected cells after they have been frozen by using essentially the same protocols set forth above.

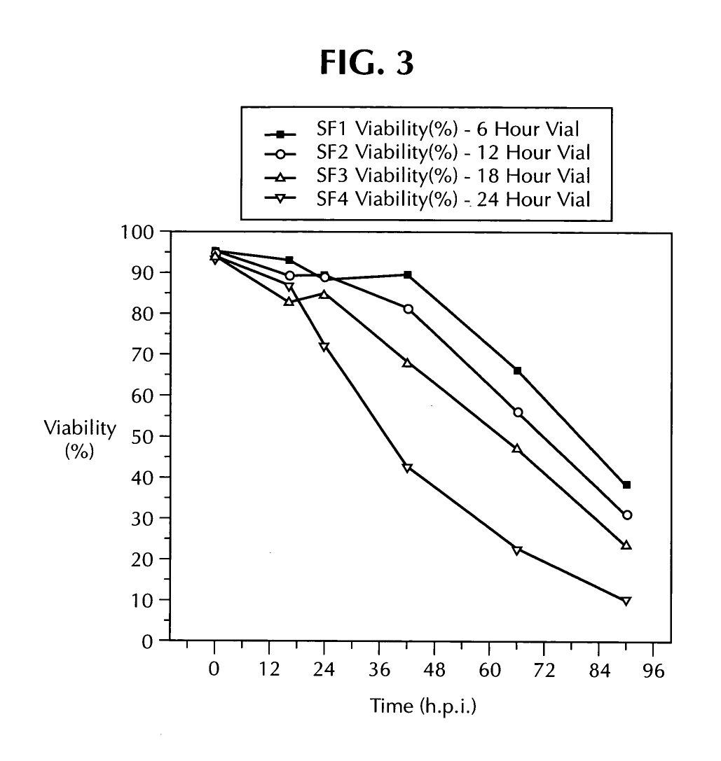

FIGS. 2 and 3 illustrate, and Example 1 describes, how the viability of Sf9 cells infected with recombinant baculovirus expressing BACE remains virtually unchanged due to the freezing process. FIG. 2 shows the viability of the incubated cells at various time points during incubation. FIG. 3 shows the viability of incubated cells captured at the 6, 12, 18, and 24 hour time points during the incubation shown in FIG. 2, after having been separated and collected, admixed with a cryo-protective agent, frozen, thawed at 37° C., and diluted 1:20 in SF 900-II SFM media, as set forth in Example 1. As can be seen when one compares the viability of the incubated cells in FIG. 2 and the viability of the cells at the zero time point in FIG. 3, the viability remains virtually unchanged although the cells have been frozen and thawed (i.e., the cell viability remains at about 95% as measured using the CEDEX apparatus).

Any method commonly used in the art for cooling can be used to freeze the admixture. Preferably, to achieve uniform, controlled cooling, a programmable-rate cell freezing apparatus is used, such as, for example, the KRYO 10 Series II manufactured by Planer Products, Ltd. Another example is the Nalgene “Cryo 1 Degree C Freezing Container” (VWR #55710-200), which can also be used to achieve a uniform, controlled cooling rate (1° C./minute).

Preferably, the admixture is frozen by reducing the temperature at a rate of from 1° C./minute to 5° C./minute, more preferably 1° C./minute. Preferably the admixture is frozen for a time and under conditions sufficient to provide that the temperature of the admixture is less than or equal to −20° C., more preferably less than or equal to −70° C., most preferably less than or equal to −130° C. The frozen admixture can then be stored in a freezer or in liquid nitrogen.

The cryogenically protected viral delivery system of the present invention comprises the frozen admixture of concentrated infected cells contained in a cryogenic vessel.

Preferably, the cells in the cryogenic vessel of the delivery systems of the invention have a viability above 50%, more preferably above 70%, most preferably above 90%.

Preferably, viability and/or average cell diameter are determined using one of the automated apparatuses set forth above for measuring cell viability and average cell diameter, such as, for example, the VI-CELL apparatus manufactured by Beckman, the PERSONAL CELL ANALYZER manufactured by Guava, or the CEDEX apparatus, manufactured by Innovatis. Most preferably, viability and/or average cell diameter are determined using the CEDEX apparatus. Typically, a sample of the frozen admixture would be removed, thawed in a 37° C. waterbath, serially diluted, and analyzed using the CEDEX apparatus.

Preferably, the virally infected cells in the cryogenic vessel of the cryogenically protected viral delivery system of the invention represent at least 20% of the total number of cells in the vessel, more preferably at least 40%, even more preferably at least 60%, most preferably at least 80%.

The percentage of virally infected cells in the cryogenic vessel can be determined by any method commonly used in the art. Preferably, the GFP method, which utilizes a recombinant version of the virus of interest that expresses GFP, is used to determine the percentage of virally infected cells. The GFP-recombinant virus is used to infect host cells that produce the virally infected cells of the cryogenically protected viral delivery system. The cells that are infected with the GFP-recombinant virus emit green light (i.e., fluorescence). A sample of the cells is then removed, thawed in a 37° C. waterbath, serially diluted, and visually analyzed under a fluorescence microscope, such as, for example, a Nikon TE 300 microscope with an FITC cube (catalog No. R620-07). The number of green light emitting cells and the total number of cells are counted, and the numbers computed to give a percentage of virally infected cells.

More preferably, the percentage of virally infected cells is determined using flow cytometry. This method involves removing a sample of the cells, thawing the sample in a 37° C. waterbath, washing the cells using PBS containing TWEEN 20, and then permeating them by treatment with a solution containing p-formaldehyde and lysis reagent, such as, for example, Becton Dickinson No. 349202. The washed and permeated cells are then incubated with fluorescin conjugated antibody that is specific for a protein expressed by the virus of interest, such as, for example, a virus nucleocapsid protein. The antibody treated cells are then analyzed using flow cytometry, preferably by using a Fluorescence Activated Cell Sorter (“FACS”), such as, for example, the Becton Dickinson FACS VANTAGE apparatus. One of skill in the art will readily recognize that antibodies that are coupled to a fluorescent agent and that are specific for a protein expressed by many viruses of interest are well known and can readily be selected for use.

The concentration of virally infected cells in the cryogenic vessel is generally from 10⁶ cells/ml to 10⁹ cells/ml, more preferably from 5×10⁶ cells/ml to 5×10⁸ cells/ml. The concentration of virally infected cells in the vessel can be determined by any method commonly used in the art, such as, for example, by removing a sample of the frozen admixture, thawing the admixture in a 37° C. waterbath, serially diluting the sample, placing a small amount of the serial dilutions on a slide, using a microscope to count the number of cells in a given volume of the diluted sample, and then mathematically calculating the concentration of total cells in the undiluted admixture. With this information and the percentage of virally infected cells determined by the GFP or flow cytometry methods set forth above, one can calculate the concentration of virally infected cells in the vessel.

Preferably, the volume of the admixture contained in the cryogenic vessel is from 0.1 ml to 200 ml, more preferably from 0.5 ml to 20 ml, most preferably from 1 ml to 4 ml. The volume of the admixture in the cryogenic vessel can be determined using any method commonly used in the art, such as, for example, by placing the admixture in a calibrated container and determining the volume by visual inspection.

Preferably, the cryogenic vessel contains from 10⁵ to 10¹² virally infected cells, more preferably from 10⁶ to 10¹⁰ virally infected cells, most preferably from 5×10⁶ to 5×10⁸ virally infected cells. The number of virally infected cells in the cryogenic vessel is preferably determined using the GFP and fluorescence microscope method set forth above, more preferably using the flow cytometry method set forth above.

Preferably the cryogenically protected viral delivery system of the present invention is substantially free of extracellular viral particles and substantially free of spent incubation media.

As used herein, “substantially free of extracellular viral particles” shall be understood to mean that the extracellular viral particles are less than 10% of the total number of viral particles present. More preferably, the extracellular particles present after the separating and collecting steps are less than 3% of the total number of viral particles present, most preferably less than 1%.

In order to calculate the percentage of the total number of viral particles that are extracellular viral particles, typically the total number of viral particles present and the number of extracellular viral particles present are determined and then the percentage is mathematically calculated.

The total number of viral particles present after the freezing step can preferably be determined by thawing the frozen admixture, such as, for example, by placing the admixture in a 37° C. waterbath, diluting the admixture to a final cell concentration of 10⁶ cells/ml, lysing the cells, and performing a p.f.u. assay on the lysate.

The number of extracellular particles present after the freezing step can preferably be determined by thawing the frozen admixture, such as, for example, by placing the admixture in a 37° C. waterbath, and gently removing the cells from the thawed admixture, such as, for example, by centrifugation or by filtering through a filter with a small enough pore size to allow the media and extracellular viral particles to pass, but not the cells, and then performing a p.f.u. assays on the separated media.

A particularly preferred embodiment of the present invention is a cryogenically protected viral delivery system wherein the virally infected cells are Sf9 cells infected with recombinant baculovirus.

Another preferred embodiment of the present invention is a cryogenically protected viral delivery system wherein the virally infected cells are Sf9 cells infected with recombinant baculovirus carrying a heterologous polynucleotide operatively linked to a baculovirus polyhedrin promoter.

A further preferred embodiment of the present invention is a cryogenically protected viral delivery system wherein the virally infected cells are mammalian cells infected with adenovirus.

Yet another preferred embodiment of the present invention is a cryogenically protected viral delivery system wherein the virally infected cells are HEK-293 cells infected with adenovirus.

A further preferred embodiment of the present invention is a cryogenically protected viral delivery system wherein the virally infected cells are HEK-293 cells infected with recombinant adenovirus.

A further preferred embodiment of the present invention is a cryogenically protected viral delivery system wherein the virally infected cells are mammalian cells infected with influenza virus.

Use of the Cryopreserved Delivery System

The cryogenically protected viral delivery systems of the invention can be used for a variety of purposes. For example, they may be used to infect host cells and consequently to produce viruses and viral products in high yields and in a consistent (i.e., reproducible) manner.

As the viability of the cells contained in the cryogenically protected viral delivery system, and the ability of the system to initiate infection, remains virtually unchanged while it is stored in a frozen state for extended periods, it is generally unnecessary to titer the delivery system prior to use.

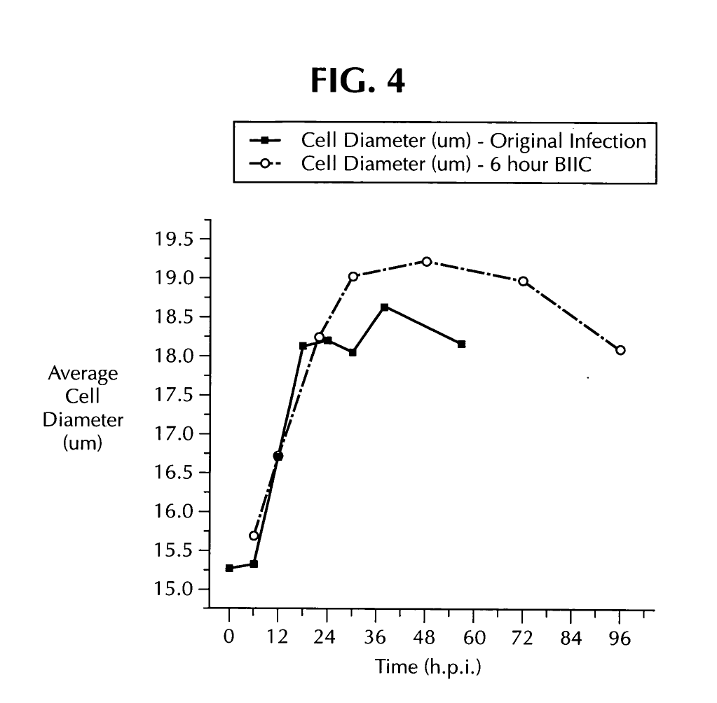

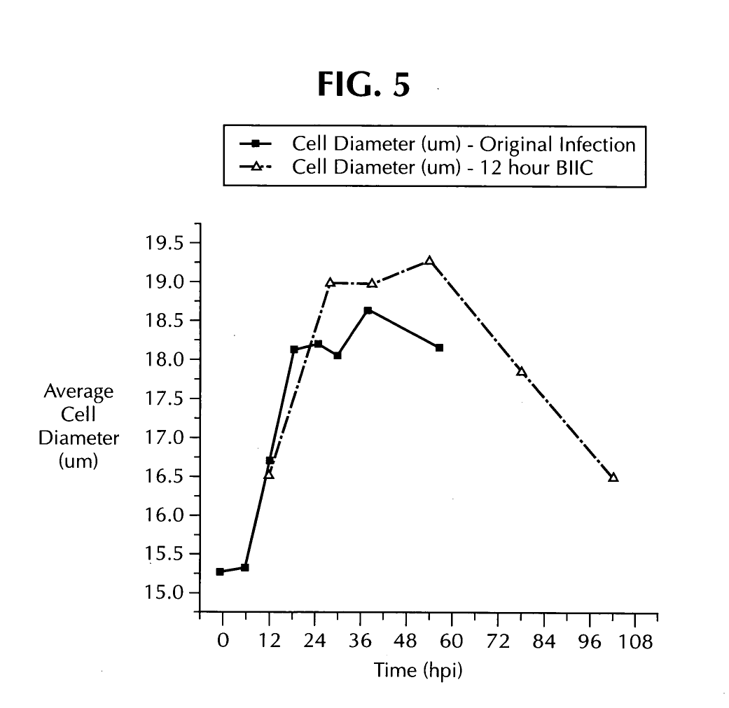

The ability of the cryogenically protected viral delivery system to initiate a productive infection is illustrated in FIGS. 4 and 5 and described in Example 1. FIGS. 4 and 5 illustrate how Sf9 cells infected with baculovirus expressing BACE captured at the 6 and 12 hour time points during incubation, after having been separated and collected, admixed with a cryo-protective agent, frozen, thawed at 37° C., diluted 1:20 in SF 900-II SFM media, and incubated again, as set forth in Example 1, continued to swell (i.e., average cell diameter increased) during incubation (T=6 and T=12 in FIGS. 4 and 5, respectively). FIGS. 4 and 5 also show the increase in cell size observed during the initial incubation of the inoculated cells, prior to collecting the cells. As can be seen, the rates of cell size increase are similar for the 6 hour, 12 hour and initial incubation samples. The similarities in the rate of cell size increase suggests that the cryogenically preserved viral delivery system is capable of picking up the infection process from the point at which the cells were collected, as though the incubation had proceeded unabated.

The cryogenically protected viral delivery system can be directly used to initiate the infection process by inoculating a plurality of uninfected host cells with the frozen admixture. Alternatively, the frozen admixture can first be thawed, either partially or completely, prior to adding to uninfected host cells. Any method commonly used in the art for thawing a cell suspension can be used, such as, for example, by placing the cryogenically protected viral delivery system in a 37° C. waterbath until the frozen admixture liquefies.

In general, the method of infecting cells involves providing a plurality of virally infected cells obtained from the admixture contained in the cryogenically protected viral delivery system, providing a plurality of uninfected host cells, preferably having a concentration of uninfected host cells from 1×10⁵ cells/ml to 1×10⁷ cells/ml, optionally thawing, washing and/or lysing the virally infected cells, and inoculating at least a portion of the uninfected host cells with at least a portion of the plurality of virally infected cells.

The concentration of uninfected host cells can be determined by any method commonly used in the art for such purpose. Preferably, the concentration of uninfected host cells is determined by removing a small sample of the cells, serially diluting the sample, placing a small amount of the serial dilutions on a slide, using a microscope to count the number of cells in a given volume of the diluted sample, and then mathematically calculating the concentration of cells in the undiluted admixture. More preferably, the concentration of uninfected host cells is determined by using an automated apparatus capable of measuring cellular concentration, such as, for example, the CEDEX apparatus.

The uninfected host cells can be any of the wide variety of preferred cell types that may be used in the present invention, as set forth above. One of skill on the art will recognize that the cell type selected will vary depending on various criteria, such as, for example, whether the cell type is susceptible to infection with the virus of interest.

The admixture used to inoculate the uninfected host cells preferably has the same characteristics as those set forth in describing the cryogenically protected viral delivery system of the present invention, and are determined using the same protocols set forth above. In particular, the concentration of virally infected cells in the admixture is preferably from 1×10⁶ cells/ml to 1×10⁹ cells/ml as determined using the flow cytometry method set forth above, the viability of the cells in the admixture is preferably at least 50%, more preferably at least 90%, as determined using the CEDEX apparatus set forth above, the admixture is at a temperature of less than or equal to −20° C., more preferably less than or equal to −70° C., and the volume of the admixture is preferably from 0.1 ml to 200 ml as determined using visual inspection in a calibrated container.

It is preferred that uninfected host cells are inoculated to provide that the number of virally infected cells added to the uninfected host cells represent no more than 10% of the total number of cells, more preferably no more than 1% of the total number of cells. The percentage of virally infected cells can be readily controlled by selecting the appropriate volume and concentration of virally infected cells, and the volume and cell concentration of the plurality of uninfected host cells, used in the inoculation.

The virally infected cells contained in the admixture of the cryogenically protected viral delivery system can optionally be washed prior to inoculating uninfected host cells. Washing can be used to remove unwanted ingredients in the admixture. Washing of the cells can be carried out using any method commonly used in the art for washing cells. Preferably the cells are washed by diluting the cells in buffer or incubation media, such as, for example, SF 900-II SFM, and then collecting and separating the cells from the buffer, such as, for example, by centrifuging the diluted cells at about 150×g for 10 minutes and decanting the supernatant. The pelleted cells can then be used to initiate the infection process, such as, for example, by admixing the pelleted cells with incubation media and using the admixed cells to inoculate uninfected host cells.

The virally infected cells contained in the admixture of the cryogenically protected viral delivery system can optionally be lysed prior to inoculating uninfected host cells. Since typically, it is the virus contained in the cryogenically protected viral delivery system that actually initiates the infection process, it generally is not detrimental to lyse the cells prior to admixing with uninfected host cells. Accordingly, the cells in the admixture can be lysed by any method commonly used in the art for lysing cells, such as, for example, by sonicating the admixture, by repeatedly freezing and thawing the admixture, by placing the cells in the admixture in a hypotonic solution, or by incubating the cells in the admixture in the presence of incubation media for sufficient time to allow the viral bursting or lytic process to proceed partially or completely. Preferably, the cells are incubated in an admixture in the presence of incubation media for sufficient time to allow the viral bursting or lytic process to proceed partially or completely.

The inoculated cells are then preferably incubated in a composition comprising incubation media for a time and under conditions sufficient so that at least 20% of the cells in the composition are virally infected, more preferably at least 50%. The percentage of virally infected cells in the composition can preferably be determined utilizing the GFP or flow cytometry methods set forth above.

In addition, it is preferred that the inoculated cells are incubated for a time and under conditions sufficient so that the concentration of viruses in the composition is from 10⁷ viruses/ml to 10¹¹ viruses/ml. The concentration of viruses in the composition can be determined by removing a sample of the composition, lysing the cells in the composition and performing a p.f.u. assay on the lysate.

Further, it is preferred that the inoculated cells are incubated for a time and under conditions sufficient so that the concentration of polypeptide in the composition is from 0.0001 mg/ml to 10 mg/ml. The concentration of recombinant polypeptide can preferably be determined by using an enzyme-linked immunosorbant assay (ELISA), such as, for example, by following the ELISA protocol set forth in D. J. Reen, Enzyme-linked Immunosorbant Assay (ELISA), Meth. Mol. Biol. 32:461-466 (1994).

Determination of the incubation conditions employed and of the media used, may be made based on factors familiar to those skilled in the art.

Production of Viral Products

Numerous types of viral products can be produced using the delivery systems and methods of the present invention, such as, for example, polypeptides and polynucleotides. The viral products can be those naturally produced by the virus or can be produced as a result of recombinantly cloned heterologous polynucleotides. Viral products include whole or partial viruses as well as heterologous or homologous polypeptides, such as, for example, proteins, enzymes, hormones, cytokines, receptors, antigens, antibodies, and fusion proteins thereof. As used herein the term “heterologous” is understood to mean other than naturally produced by the virus or host cell of interest and the term “homologous” is understood to mean naturally produced by the virus or host cells of interest.

The heterologous polynucleotide carried by the recombinant virus of the present invention can be operatively linked to any heterologous or homologous promoter that is commonly used in the art to drive the transcription and/or translation of a heterologous polynucleotide. Preferably, the promoter is either a CMV, CMV-IE, TK, SV40, T7, Sp6, EM7, bla, Actin, MT (metallothionein), EF-1 alpha, TET, an ecdysteroid responsive promoter, MMTV, HSV, HSV-IE 175, MuLV, RSV, EF-1, or a baculovirus promoter, such as, for example, the baculovirus polyhedrin (i.e., polh), p10, 35 kd protein, XIV, (late) basic protein, (early) ETL, core protein (cor), immediate early (e.g., IEO, IE1, IE-N, and pe38), and (delayed early) DE promoter. More preferably the promoter is the baculovirus polyhedrin, CMV, CMV-IE, SV40, EF-1 alpha, MT, or TET promoter. Most preferably, the promoter is the baculovirus polyhedrin promoter. As used herein, the term “operatively linked” is understood to mean that the promoter is sufficiently connected to the heterologous polynucleotide to direct and regulate its transcription and/or translation.

The viral products produced using the method of the present invention can be isolated/harvested/collected using one of the many protocols commonly used in the art.

Preferably, viruses can be isolated by first lysing virally infected cells to release virus and then harvesting the virus. Lysis of the cells can be carried out using a variety of methods commonly used in the art, such as, for example, the addition of a small amount of chloroform to the cells and mixing the resulting suspension. One should note that it is possible that some lysis of cells might have already occurred as a natural consequence of the infection process, therefore, some virus might already be present in solution. In addition, some virus types can exit the host cells without necessarily lysing the cell. Accordingly, lysing of the virally infected cells might not be necessary due to sufficient virus being present in solution as a result of cells already having been lysed by the virus or the natural secretion of virus.

Once the virally infected cells are lysed, the virus can be separated from cellular debris and any remaining cells by any method commonly used in the art, such as, for example, by centrifuging the solution at 10,000×g for 10 minutes twice. The partially purified virus can then be collected by any method commonly used in the art for collecting virus from solution, such as, for example, by centrifuging the resulting supernatant at a higher speed, such as, for example, at 40,000×g for 30 minutes, to collect a partially purified viral pellet. The virus in the resuspended pellet can then be further purified by any method commonly used in the art, such as, for example, by resuspending the pellet in buffer, such as, for example, TE buffer, and centrifuging the resuspended pellet through a sucrose step gradient, such as for example by centrifugation at 40,000×g for 30 minutes through a 5%/40% sucrose step gradient. In a 5%/40% sucrose step gradient, the virus collects between the two layers of sucrose and is harvested by removing the collected virus with a pipette.

Preferably, viral products other than viruses that are not secreted by the infected cells can be isolated by collecting the infected cells using any method commonly used in the art for this purpose, such as, for example, by spinning down the cells at 2,500×g for 5 minutes, lysing the cells by incubating the cells in a buffer that contains a detergent that is known in the art to lyse cells, such as, for example, 5% Triton-X100, clearing the cellular debris by centrifuging at 45,000×g for 30 minutes and collecting the supernatant, which should contain the viral product of interest.

Preferably, viral products that are secreted can be isolated by simply centrifuging the culture for 2,500×g for 5 minutes to pellet the cells and then collecting the supernatant which should contain the secreted viral product of interest.

The following examples are provided to further illustrate the processes and compositions of the present invention. These examples are illustrative only and are not intended to limit the scope of the invention in any way.

### EXAMPLE 1

### Preparation of the Cryogenically Protected Viral Delivery System Using Sf9 Insect Cells Infected with the Baculovirus Expression System Expressing the his-Tagged Beta-Site APP (Amyloid Precursor Protein) Cleaving Enzyme (BACE)

A recombinant baculovirus that expresses the his-tagged Beta-site APP (Amyloid Precursor Protein) Cleaving Enzyme (BACE) protein (BACE is described, inter alia, in U.S. Published Patent Application No. 2002/0049303, and International Published Document Nos. WO0047618, WO0100663, WO0017369 and WO0058479, which are owned by parties other than the owner of the current application) was constructed using the BAC-TO-BAC Baculovirus Expression Systems (Gibco #10608-016). A virus stock of 4.7×10⁸ p.f.u./ml was generated.

The Sf9 immortalized cell line was obtained from Invitrogen.

Sf9 cells were grown to 7.5×10⁵ viable cells/ml in a flask containing 200 ml of the media set forth below:

Media:

SF900 II SFM (Gibco # 10902-088)

10% (v/v) FBS (fetal bovine serum)

5 mg/L Gentamicin (Gibco #15710-072)

The cell culture was then infected with the titered baculovirus-BACE stock at an M.O.I. of 3 and incubated at 27° C., no CO₂ and humidity controls, and 130 rpm. The concentration of extracellular baculovirus and cell size (i.e., average cell diameter) (see FIG. 1), as well as the cell viability (see FIG. 2) were monitored.

Cell viability and cell size were determined by using the CEDEX assay procedure.

The concentration of extracellular baculovirus was determined by using the plaque forming units (p.f.u.) assay procedure.

After about 6 hours, 12 hours, 18, hours, 24 hours, 30 hours, 38 hours, and 50 hours of incubation, about 20 ml, 17 ml, 14 ml, 13 ml, 13 ml, 13 ml, and 17 ml of culture were removed, respectively, placed in 50-ml conical centrifuge tubes and spun down at 680 rpm for 10 minutes. The supernatant was decanted and stored at a refrigerated temperature for conducting plaque and other assays. The pellets were then resuspended in freezing media to give a viable cell concentration of about 1×10⁷ cells/ml.

Freezing Media:

90% (v/v) Sf900 II SFM (Gibco #10902-088)

1% (w/v) Bovine Serum Albumin

10% (v/v) DMSO

1 ml aliquots of the cell suspension were then pipetted into sterile 1.8 ml cryo-vials from Nalge Nunc International, Nunc Brand (377267) VWR cat. no. 66021-986. The vials were then placed in a 1° C./minute Cryo Freezing Container (Nalgene #5100-0001), and then the container was placed into a −75° C. freezer overnight. The vials were then placed into a liquid nitrogen storage tank at about −150° C. for long term storage.

One of each of the 6-hour, 12-hour, 18-hour, and 24-hour cryo-preserved vials was removed from liquid nitrogen storage, quickly thawed in a 37° C. waterbath, and diluted 1:20 in SF 900-II SFM media and the viability was monitored by the CEDEX method. FIG. 3 shows that these cryo-preserved infected cells were recovered without much loss of their viability. It's also interesting to note from FIGS. 4 and 5 that the infected cells caught at 6 hours and 12 hours post infection, spun down, resuspended in freezing media, frozen down and stored in liquid nitrogen could essentially resume their infection process as well as if the infection process had not been interrupted. FIGS. 4 and 5 used the cell diameter increase to illustrate this point. In addition, one of the 24-hour vials was removed from liquid nitrogen freezer, and quickly thawed in a 37° C. water bath. The thawed viral delivery cell suspension was added to 100 ml of SF900 II SFM media (1:100 dilution). 1 ml of the diluted cell suspension was added to each of three culture flasks containing an Sf9 cell suspension at the following concentrations in 100 ml of SF900 II SFM media:

Flask 1: 0.50×10⁶ Sf9 cells/ml

Flask 2: 0.75×10⁶ Sf9 cells/ml

Flask 3: 1.00×10⁶ Sf9 cells/ml

The resulting suspension was incubated at 27° C., no CO₂ and humidity controls, and 130 rpm. 1.5 ml aliquots were removed at 24, 48, 72, 96 and 123 hours post-infection. The aliquots were centrifuged at 16,000×g for 1 minute, the supernatant decanted and stored at −20° C. The supernatant was then thawed and analyzed via Western blot analysis using a tetra-his antibody (Qiagen # 4670). The 72-hour and 96-hour supernatants from Flask 1, Flask 2, and Flask 3 all showed BACE expression that was equal to or better than a control sample. The control sample represents the best BACE expression resulting from infections of uninfected Sf9 cells or Hi-Five cells using traditional virus stocks (i.e., extracellular virus stocks). Comparable BACE expression was also obtained when uninfected Hi-Five cells were infected with the cryogenically protected, BACE baculovirus infected Sf9 cells.

### EXAMPLE 2

### Preparation of the Cryogenically Protected Viral Delivery System Using Baculovirus Infected Sf9 Cells that Express the Truncated his-FAKcd Polypeptide

A recombinant baculovirus that expresses a portion of the cd (catalytic domain) of the FAK (focal adhesion kinase) protein was constructed using the BAC-TO-BAC baculovirus Expression Systems (Gibco #10608-016). A virus stock of 1.3×10⁸ p.f.u./ml was generated.

The Sf9 immortalized cell line was obtained from Invitrogen.

Sf9 was grown in SF-900 II SFM (Gibco #10902-088) incubation media supplemented with 0.5 ml/L gentamicin (Gibco #15710-064). 100 ml of Sf9 cells at 5×10⁵ cells/ml were inoculated with 38 microliters of the virus stock.

The inoculated cells were incubated under the following conditions: 27° C., no CO₂ and humidity controls, and 130 rpm. Cell density, cell viability, and cell diameter (which can be used to calculate cell volume) were monitored using a CEDEX unit from Innovatis.

After about 28.5 hours of incubation, when the cell size increased and plateaued and the cell viability was about 95%, 50 ml portions of the cells were placed in 50-ml conical centrifuge tubes and centrifuged at 600 rpm in a Beckman-Coulter TJ-25 centrifuge for 10 minutes. The supernatant containing the extracellular viral particles and the spent incubation media was then decanted.

The pellet was then re-suspended in a freezing medium containing 90% (v/v) SF-900 II SFM, 1% (w/v) bovine serum albumin, and 10% (v/v) DMSO, and then aliquoted into Nunc brand 1.8-ml CRYOTUBES, each aliquot having a volume of 1 ml and containing about 1×10⁷ viable cells, all steps carried out aseptically.

The vials containing the baculovirus infected Sf9 cells were then frozen down at a rate of about one ° C. per minute to a final temperature of about −75° C., and then the vials were moved into a liquid nitrogen canister for long-term storage.

One vial was retrieved from liquid nitrogen storage, thawed at 37° C., and added to 100 ml of Sf-900 II SFM media supplemented with 0.5 ml/L gentamicin. The entire flask was then added to 10 liters of uninfected Sf9 cells grown in SF-900 II SFM (Gibco #10902-096) supplemented with 0.5 ml/L gentamicin (Gibco #15710-064) at a cell density of 1.1×10⁶ cells/ml. The cells were then incubated under the following conditions: 27° C., 1 liter/minute top air, 120 rpm, D.O. controlled above 50% of saturation with sparged oxygen, pH not controlled. The entire 10-L culture was harvested after 70 hours of incubation. A control 10-L Sf9 culture grown in the same media was infected at a cell density of 1.9×10⁶ cells/ml with the conventional virus stock with an M.O.I. of 1.0. It was harvested at 68.5 hours post infection. Upon parallel purifications, the two 10-L harvests yielded 25 and 10 mg of purified FAKcd respectively.

### EXAMPLE 3

### Purification of the Truncated his-FAK Polypeptide Produced Using the Cryogenically Protected Viral Delivery System of Example 3

130 g of cell paste derived from an Sf9 cell culture inoculated with the cryogenically protected viral delivery system of Example 2 (i.e., baculovirus infected Sf9 Cells that express the truncated his-FAKcd recombinant polypeptide) was resuspended in 3 volumes (400 ml) buffer A, containing 50 mM HEPES pH 7.0, 500 mM NaCl, 0.1 mM TCEP and COMPLETE protease inhibitor cocktail tablets (Roche). The cells were lysed with one pass on a microfluidizer and the cell debris was removed by centrifugation at 4° C. for 35 minutes at 14,000 rpm in a Sorval SLA-1500 rotor. The supernatant was transferred to a clean tube and 6.0 ml of Ni-NTA agarose (Qiagen) was added. The suspension was incubated with gentle rocking at 4° C. for 1 hour and then subjected to centrifugation at 700×g in a swinging bucket rotor. The supernatant was discarded and the agarose was resuspended in 20.0 ml of buffer A and transferred to an XK-16 column (Amersham-Pharmacia) connected to an FPLC. The agarose was washed with 5 column volumes of buffer A and eluted off the column with a step gradient of buffer A containing 300 mM imidizole. The eluted fractions were buffer exchanged into buffer B containing 25 mM HEPES pH 7.0, 400 mM NaCl and 0.1 mM TCEP. Following buffer exchange, thrombin was added to the pooled fractions at a 1:300 (w/w) ratio and incubated overnight at 13° C. to remove the N-terminal his-tag. The reaction mixture was loaded back onto the Ni-NTA column equilibrated with buffer A and the flow-through was collected. The flow-through was concentrated down to 1.7 ml and loaded directly onto a SUPERDEX 200 HILOAD 16/60 prep grade column equilibrated with buffer C containing 10 mM HEPES pH 7.5, 200 mM ammonium sulfate and 0.1 mM TCEP. The desired protein eluted between 85-95 ml. The protein was aliquoted and stored frozen at −75° C.

### EXAMPLE 4

### Preparation of the Cryogenically Protected Viral Delivery System Using Baculovirus Infected Sf9 Cells that Express the PDE-1B-13 Polypeptide

Baculovirus that expresses a portion of the catalytic region of the PDE-1B protein (designated as PDE-1B-13) was constructed using the BAC-TO-BAC Baculovirus Expression Systems (Gibco #10359-016). A virus stock was generated, but not titered.

Sf9 was obtained from Invitrogen. Cell density, cell viability, and cell diameter were determined using a CEDEX unit from Innovatis.

Sf9 was grown in SF-900 II SFM (Gibco #10902-088) incubation media supplemented with 5 ml/L Antibiotic/Antimycotic (Gibco #15240-062). One liter of Sf9 cells at 1.68×10⁶ cells/ml was infected with 20 milliliters of the stock virus. After 24 hours of incubation, when the cell size increased by about 3.8 um and the cell viability was at 88.8%, the cells were spun down, supernatant decanted, pellet re-suspended in a 2× freezing medium containing 80% (v/v) SF-900 II SFM, 2% (w/v) bovine serum albumin, and 20% (v/v) DMSO, and dispensed into 1.0 ml aliquots each containing about 1×10⁸ viable cells/ml, all steps carried out aseptically. The vials containing the baculovirus infected Sf9 cells were then frozen down at a rate of about one ° C. per minute to about −75° C. and then moved into a liquid nitrogen canister for long-term storage.

Two of the frozen vials were retrieved from liquid nitrogen storage, thawed at 37° C., and the content of one and one-half vials added to 400 ml of ESF921 media (Expression Systems # 96-001-10) supplemented with 10 ml/L Antibiotic/Antimicotic (Gibco #15240-062). The entire flask was then added to 100 liters of uninfected Sf9 cells grown in ESF921 media (Expression Systems # 96-001-10) supplemented with 10 ml/L Antibiotic/Antimicotic (Gibco #15240-062) at a cell density of 1.54×10⁶ cells/ml. The entire 100-L culture was harvested after 72 hours of incubation. The expression level and recovery yield of PDE-1B-13 from the 100-L harvest are comparable to those of smaller scale infections in shake flasks, 10-L stirred tanks, or 10-L Wave bioreactors, infected with either conventional baculovirus preparations or Sf9 cells infected with baculovirus expressing PDE-1B-13.

### EXAMPLE 5

### Preparation of the Cryogenically Protected Viral Delivery System Using Baculovirus Infected Sf9 Cells that Express the Truncated GSK3-Beta Polypeptide

Baculovirus that expresses a truncated portion of the GSK3-beta protein was constructed using the BAC-TO-BAC Baculovirus Expression Systems (Gibco #10608-016). A virus stock was generated, but not titered.

Sf9 was obtained from Invitrogen. Cell density, cell viability, and cell diameter were determined using a CEDEX unit from Innovatis.

Sf9 was grown in SF-900 II SFM (Gibco #10902-088) supplemented with 0.5 ml/L gentamicin (Gibco #15710-064). 1000 ml of Sf9 cells at 1.92×10⁶ cells/ml was infected with 1 ml of the stock virus. After about 36 hours of incubation, when the cell size increased and plateaued, the cells were spun down, supernatant decanted, pellet re-suspended in a freezing medium containing 90% (v/v) SF-900 II SFM, 1% (w/v) bovine serum albumin, and 10% (v/v) DMSO, and dispensed into 1 ml aliquots each containing about 1×10⁷ cells/ml, all steps carried out aseptically. The vials containing the baculovirus infected Sf9 cells were then frozen down at a rate of about one ° C. per minute to about −75° C. and then moved into a liquid nitrogen canister for long-term storage.

One of the frozen vials was retrieved from liquid nitrogen storage, thawed at 37° C., and added to 100 ml of SF-900 II SFM (Gibco #10902-088) incubation media supplemented with 0.5 ml/L gentamicin (Gibco #15710-064). The entire flask was then added to 10 liters of uninfected Sf9 cells grown in SF-900 II SFM (Gibco #10902-088) supplemented with 0.5 ml/L gentamicin (Gibco #15710-064) at a cell density of 1×10⁶ cells/ml. The 10-L culture was harvested after 72 hours of incubation. The expression of the truncated GSK3-beta protein from the 10-L harvest was comparable to that in shake flasks, in which either Sf9 or Sf21 cells were infected with either conventional baculovirus preparations or insect cells infected with baculovirus expressing the truncated GSK3-beta.

### EXAMPLE 6

### Preparation of the Cryogenically Protected Viral Delivery System Using Adenovirus Infected HEK-293 Cells that Express the GFP Polypeptide

An Ad5.CMV-GFP adenovirus stock was obtained from Q-Biogene (catalog number ADV0030). This is a recombinant virus that expresses the GFP protein. The manufacturer's specifications indicated a virus titer of about 1-1.5×10¹¹ viral particles/ml.

The HEK (human embryonic kidney)-293 immortalized cell line, 293-EBNA, was obtained from Invitrogen (catalog number R620-07).

293-EBNA was grown in 293 SFM II (Gibco cat. No. 11686) incubation media supplemented with 5% fetal bovine serum, 4 mM L-glutamine, and 250 mg/L Geneticin. 100 ml of 293-EBNA cells at 7.5×10⁵ cells/ml were inoculated with 37.5 microliters of the Ad5.CMV-GFP (ADV0030) virus stock. The projected M.O.I. was 50-75.

The inoculated cells were incubated under the following conditions: 36.5° C., 5% CO₂, 75% humidity, and 130 rpm. Cell density, cell viability, and cell diameter (which can be used to calculate cell volume) were monitored using a CEDEX unit from Innovatis. The percentage of virally infected cells was determined by visually measuring the percentage of cells emitting green fluorescence due to the expression of GFP using a Nikon TE 300 microscope with an FITC cube (part number FITC HQ C-27766) fluorescence microscope.

As the cell size increased and plateaued and when greater than 60% of the cells were GFP positive and the cell viability was about 69%, 30 ml portions of the cells were placed in 50-ml conical centrifuge tubes and centrifuged at about 150×g for 5 minutes. The supernatant containing the extracellular viral particles and the spent incubation media was then decanted.

The pellet was then re-suspended in a freezing medium containing 70% (v/v) growth media, 20% (v/v) fetal bovine serum, and 10% (v/v) DMSO to obtain a concentration of virally infected cells of 0.9×10⁷ cells/ml, and then aliquoted into 1.8 ml cryovials from Nalge Nunc International, Nunc Brand #377267 (VWR #66021-986), each aliquot having a volume of 1 ml and containing about 1×10⁷ viable cells.

The vials containing the adenovirus infected 293-EBNA cells were then frozen down at a rate of about 1° C. per minute to a final temperature of about −75° C., and then the vials were moved into a liquid nitrogen canister for long-term storage. The viability of the cells in the vial was determined after freezing to be about 52% by using the CEDEX assay. The concentration of virally infected cells was determined to be about 0.9×10⁷ cells/ml by using the GFP method. The percentage of total cells that are virally infected cells was determined to be about 60% by the GFP method.

One vial of the frozen adenovirus infected 293-EBNA cells was then retrieved from liquid nitrogen storage, thawed at about 37° C., and added to 100 ml of 293 SFM-II media supplemented with 5% fetal bovine serum, 4 mM L-glutamine, and 250 mg/L Geneticin to create a “master virus flask (MVF).” The cells were then incubated under the following conditions: 36.5° C., 5% CO₂, 75% humidity, and 130 rpm. Following incubation for 0, 24, 48, or 72 hours, a portion from the MVF was added to uninfected 293-EBNA cells at a 1:10 ratio to create “intermediate virus flasks (IVF).” Following 72 hours of incubation, a portion of the IVF was added to uninfected 293-EBNA cells at a 1:100 ratio to create the “final virus flasks (FVF).” Time point samples were taken from MVF, IVF, and FVF for cell density, cell viability, cell diameter, and GFP determinations. Under preferred conditions, in which the MVF was incubated for 48 or 72 hours, a maximum of 80-90% cells were virally infected as determined by visually measuring the percentage of cells emitting green fluorescence due to the expression of GFP using a Nikon TE 300 microscope with an FITC cube (part number FITC HQ C-27766) fluorescence microscope after 48 hours of incubation in the FVF. The control condition, that is, when a commercially available and traditional Ad5.CMV-GFP virus preparation was used to infect 293-EBNA cells directly with an M.O.I. of 50-75, a maximum of 75-80% cells were GFP positive at 24 hours.

### EXAMPLE 7

### Preparation of the Cryogenically Protected Viral Delivery System Using Baculovirus Infected Sf9 Cells that Express the G Protein

Recombinant baculoviruses that express the alpha, beta, and gamma subunits of the G protein were obtained from BioSignal (catalog numbers BCV-106, BCV-105, and BCV-161 respectively). The manufacturer's specifications indicated that the virus titers of the virus stocks were about 2-5×10⁸ p.f.u./ml. The commercial viruses were first expanded in volume, following a standard baculovirus expansion method. See Gibco BRL Instruction Manual, Guide to Baculovirus Expression Vector System (BEVS) and Insect Cell Culture Techniques (2002). The newly expanded virus stocks for the alpha, beta, and gamma subunits had titers of 7, 10, and 8×10⁸ p.f.u./ml, respectively.

The Sf9 immortalized cell line was obtained from invitrogen.

Sf9 was grown in SF-900 II SFM (Gibco cat. 10902-088) supplemented with 5 mg/L gentamicin. 220 ml of Sf9 cells at 5×10⁵ cells/ml were inoculated with 73.3 microliters of the expanded baculovirus stock expressing the alpha subunit of the G protein.

The inoculated cells were incubated under the following conditions: 27° C., no CO₂ and humidity controls, and 130 rpm. Cell density, cell viability, and cell diameter (which can be used to calculate cell volume) were monitored using a CEDEX unit from Innovatis.

After about 46 hours of incubation, when the cell size increased from about 14 to about 18-19 microns and the cell viability was above 90%, 50 ml portions of the cells were placed in 50-ml conical centrifuge tubes and centrifuged at 680 rpm in a Beckman CPR centrifuge for 10 minutes. The supernatant containing the extracellular viral particles and the spent incubation media was then decanted.

The pellet was then re-suspended in a freezing medium containing 90% SF-900 II SFM, 1% (w/v) bovine serum albumin, and 10% DMSO, and then aliquoted into Nunc brand 1.8-ml CRYOTUBES, each aliquot having a volume of 1.1 ml and containing about 1×10⁷ viable cells, all steps carried out aseptically.

The vials containing the baculovirus infected Sf9 cells (BIIC-A) were then frozen down at a rate of about 1° C. per minute to a final temperature of about −75° C., and then the vials were moved into a liquid nitrogen canister for long-term storage.

Using the same protocol set forth above, vials containing virally infected cells infected with baculovirus expressing either the beta or gamma subunits of the G protein (BIIC-B and BIIC-G, respectively) were also prepared. In addition, vials containing virally infected cells infected with baculoviruses expressing the alpha, beta and gamma subunits of the G protein (BIIC-ABG) were prepared similarly except that 73.3 microliters of each of the three viruses (alpha, beta, and gamma) were used to infect Sf9 cells.

One vial of each of the BIIC-A, BIIC-B, BIIC-G, and BIIC-ABG virally infected cells was then retrieved from liquid nitrogen storage, thawed at 37° C., and 1.0 ml was added to 100 ml of Sf900-II SFM media supplemented with 5 mg/L gentamicin to create diluted A, B, G, and ABG flasks. The cells were then incubated under the following conditions: 27° C., no CO₂ and humidity controls, and 130 rpm. After about 5 minutes of incubation, 1 ml from each of the diluted A, B, and G flasks was added to 100 ml of uninfected Sf9 cells grown in IPL-41 media (Gibco #11405081) supplemented with 2% inactivated fetal bovine serum (Gibco #16140-071), 1% lipid concentrate (Gibco #21900-030), and 1% antibiotic/antimycotic (Gibco #15240-062) at a cell density of 9.67×10⁵ cells/ml to create the A+B+G final flask. Also, 1 ml from the diluted ABG flask was added to 100 ml of uninfected Sf9 cells grown in the same media at the same cell density to create the ABG final flask. Time point samples were taken from the A+B+G and ABG final flasks for analyzing cell density, cell viability, cell diameter, and western analysis for the expression of the G protein. The 72-hour and 96-hour samples from either the A+B+G or the ABG final flask showed comparable production of immunoreactive G protein. The control was obtained when the conventional virus stocks expressing the alpha, beta, and gamma subunits were used to co-infect uninfected Sf9 cells.

We claim:

1. A cryogenically protected viral delivery system for infecting host cells comprising a cryogenic vessel and a plurality of virally infected cells in admixture with a cryo-protective agent contained in the cryogenic vessel,
wherein the concentration of virally infected cells is from 10⁶ cells/ml to 10⁹ cells/ml;
wherein the admixture of the virally infected cells and the cryo-protective agent is at a temperature of less than or equal to −20° C.; and
wherein the viability of cells contained in the cryogenic vessel is at least 50%.

2. The cryogenically protected viral delivery system according to claim 1, wherein the viability of the cells is at least 70%.

3. The cryogenically protected viral delivery system according to claim 1, wherein the viability of the cells is at least 90%.

4. The cryogenically protected viral delivery system according to claim 1, wherein the admixture is substantially free of extracellular viral particles.

5. The cryogenically protected viral delivery system according to claim 1, wherein the admixture is substantially free of spent incubation media.

6. The cryogenically protected viral delivery system according to claim 1, wherein the average cell diameter of cells contained in the cryogenic vessel is at least 0.5 μm greater than the average cell diameter of uninfected cells of the same type.

7. The cryogenically protected viral delivery system according to claim 1, wherein the admixture of virally infected cells and cryo-protective agent is at a temperature of less than or equal to −70° C.

8. The cryogenically protected viral delivery system according to claim 1, wherein the admixture of virally infected cells and cryo-protective agent is at a temperature of less than or equal to −130° C.

9. The cryogenically protected viral delivery system according to claim 1, wherein the cryo-protective agent is selected from the group consisting of DMSO, serum albumin, serum, glycerol, and mixtures thereof.

10. The cryogenically protected viral delivery system according to claim 1, wherein the cryogenic vessel contains from 10⁵ to 10¹² virally infected cells.

11. The cryogenically protected viral delivery system according to claim 1, wherein the volume of the cryogenic vessel is less than or equal to 250 ml.

12. The cryogenically protected viral delivery system according to claim 1, wherein the volume of the cryogenic vessel is less than or equal to 30 ml.

13. The cryogenically protected viral delivery system according to claim 1, wherein the volume of the cryogenic vessel is less than or equal to 6 ml.

14. The cryogenically protected viral delivery system according to claim 1, wherein the cryogenic vessel is a polypropylene vial having a volume of less than or equal to 6 ml.

15. The cryogenically protected viral delivery system according to claim 1, wherein the virally infected cells in the vessel represent at least 20% of the total number of cells in the vessel.

16. The cryogenically protected viral delivery system according to claim 1, wherein the virally infected cells in the vessel represent at least 40% of the total number of cells in the vessel.

17. The cryogenically protected viral delivery system according to claim 1, wherein the virally infected cells in the vessel represent at least 60% of the total number of cells in the vessel.

18. The cryogenically protected viral delivery system according to claim 1, wherein the virally infected cells are eukaryotic cells.

19. The cryogenically protected viral delivery system according to claim 1, wherein the virally infected cells are insect cells.

20. The cryogenically protected viral delivery system according to claim 1, wherein the virally infected cells are infected with recombinant virus.

21. The cryogenically protected viral delivery system according to claim 1, wherein the virally infected cells are infected with a virus selected from the group consisting of baculovirus, adenovirus, adeno-associated virus, influenza virus, canarypox virus, infectious bovine rhinotracheitis virus, bovine viral diarrhea virus, parainfluenza 3 virus, bovine respiratory syncytial virus, feline calicivirus, chlamydia virus, canine coronavirus, panleukopenia virus, feline leukemia virus, hepatitis A, hepatitis B, hepatitis C, human immunodeficiency virus type 1 (HIV-1) and type 2 (HIV-2), cytomegalovirus, human T-lymphotropic virus type I (HTLV-I) and type II (HTLV-II), encephalitis virus, measles virus, mumps virus, rubella virus, polio virus, rabies virus, respiratory syncytial virus, rotavirus, smallpox virus, typhoid vaccine virus, varicella virus, yellow fever vaccine virus, and combinations thereof.

22. The cryogenically protected viral delivery system according to claim 1, wherein the virally infected cells are infected with baculovirus.

23. The cryogenically protected viral delivery system according to claim 1, wherein the virally infected cells are infected with adenovirus.

24. The cryogenically protected viral delivery system according to claim 1, wherein the virally infected cells are infected with adeno-associated virus.

25. The cryogenically protected viral delivery system according to claim 1, wherein the virally infected cells are infected with influenza virus.

26. The cryogenically protected viral delivery system according to claim 1, wherein the virally infected cells are Sf9 cells infected with recombinant baculovirus.

27. The cryogenically protected viral delivery system according to claim 1, wherein the virally infected cells are Sf9 cells infected with recombinant baculovirus carrying a heterologous polynucleotide operatively linked to a baculovirus polyhedrin promoter.

28. The cryogenically protected viral delivery system according to claim 1, wherein the virally infected cells are mammalian cells infected with adenovirus.

29. The cryogenically protected viral delivery system according to claim 1, wherein the virally infected cells are HEK-293 cells infected with adenovirus.

30. The cryogenically protected viral delivery system according to claim 1, wherein the virally infected cells are HEK-293 cells infected with recombinant adenovirus.

31. The cryogenically protected viral delivery system according to claim 1, wherein the virally infected cells are mammalian cells infected with influenza virus.

32. An intermixture of virally infected cells and uninfected host cells obtained by:
providing a plurality of frozen virally infected cells obtained from the admixture contained in the cryogenically protected viral delivery system of claim 1;
providing a plurality of uninfected host cells, wherein the concentration of the uninfected host cells is from 10⁵ cells/ml to 10⁷ cells/ml;
optionally thawing, washing and/or lysing the virally infected cells; and
inoculating at least a portion of the uninfected host cells with at least a portion of the virally infected cells.

33. The intermixture according to claim 32, wherein the viability of cells obtained from the admixture contained in the cryogenically protected viral delivery system of claim 1 is at least 70%.

34. The intermixture according to claim 32, wherein the viability of cells obtained from the admixture contained in the cryogenically protected viral delivery system of claim 1 is at least 90%.

35. The intermixture according to claim 32, wherein the temperature of the virally infected cells obtained from the admixture contained in the cryogenically protected viral delivery system of claim 1 is less than or equal to −70° C.

36. The intermixture according to claim 32, wherein the temperature of the virally infected cells obtained from the admixture contained in the cryogenically protected viral delivery system of claim 1 is less than or equal to −130° C.

37. A method for preparing a cryogenically protected viral delivery system comprising:
admixing a plurality of virally infected cells with a cryo-protective agent to obtain an admixture having a concentration of virally infected cells of from 10⁶ cells/ml to 10⁹ cells/ml; and
freezing at least a portion of the admixture for a time and under conditions sufficient so that the temperature of the frozen admixture is less than or equal to −20° C. and so that the viability of the cells in the frozen admixture is at least 50%.

38. The method according to claim 37, wherein at least a portion of the admixture is frozen for a time and under conditions sufficient so that the viability of the cells in the frozen admixture is at least 70%.

39. The method according to claim 37, wherein at least a portion of the admixture is frozen for a time and under conditions sufficient so that the viability of the cells in the frozen admixture is at least 90%.

40. The method according to claim 37, wherein the admixture is substantially free of extracellular viral particles.

41. The method according to claim 37, wherein the admixture is substantially free of spent incubation media.

42. The method according to claim 37, wherein at least a portion of the admixture is frozen for a time and under conditions sufficient so that the temperature of the frozen admixture is less than or equal to −70° C.

43. The method according to claim 37, wherein at least a portion of the admixture is frozen for a time and under conditions sufficient so that the temperature of the frozen admixture is less than or equal to −130° C.

44. The method according to claim 37, wherein at least a portion of the admixture is frozen by reducing the temperature at a rate of from 1° C./minute to 30° C./minute.

45. The method according to claim 37, wherein the plurality of virally infected cells and the cryo-protective agent are admixed in amounts to obtain an admixture having a concentration of virally infected cells of from 5×10⁶ cells/ml to 5×10⁸ cells/ml.

46. The method according to claim 37, wherein the plurality of virally infected cells are admixed with a cryo-protective agent selected from the group consisting of DMSO, serum albumin, serum, glycerol and mixtures thereof.

47. The method according to claim 37, further comprising preparing the plurality of virally infected cells by:
inoculating a plurality of uninfected host cells with a plurality of viruses;
incubating the inoculated cells in a composition comprising incubation media for a time and under conditions sufficient to obtain a plurality of virally infected cells; and
separating the plurality of virally infected cells from substantially all spent incubation media and extracellular viral particles and collecting the plurality of virally infected cells in a vessel.

48. The method according to claim 47, wherein the plurality of virally infected cells are collected in a vessel having a size of at least 100 ml.

49. The method according to claim 37, further comprising aliquoting at least a portion of the admixture into cryogenic vessels prior to freezing the aliquoted admixture.

50. The method according to claim 49, wherein at least a portion of the admixture is aliquoted into cryogenic vessels having a volume of less than or equal to 6 ml.

51. The method according to claim 50, wherein the cryogenic vessels are polypropylene vials.

52. The method according to claim 49, wherein at least a portion of the admixture is aliquoted into the cryogenic vessels in an amount from 10⁶ to 10⁹ virally infected cells.

53. The method according to claim 49, wherein the volume of the admixture aliquoted into the cryogenic vessels is from 0.5 ml to 20 ml.

54. The method according to claim 37, wherein the virally infected cells are eukaryotic cells.

55. The method according to claim 37, wherein the virally infected cells are insect cells.

56. A method of virally infecting cells comprising:
providing a plurality of virally infected cells in admixture with a cryo-protective agent contained in a cryogenic vessel;
wherein the concentration of virally infected cells is from 10⁶ cells/ml to 10⁹ cells/ml;
wherein the admixture of the virally infected cells and the cryo-protective agent is at a temperature of less than or equal to −20° C.; and
wherein the viability of cells contained in the cryogenic vessel is at least 50%;
providing a plurality of uninfected host cells, wherein the concentration of uninfected host cells is from 10⁵ cells/ml to 10⁷ cells/ml;
optionally thawing, washing and/or lysing the virally infected cells;
inoculating at least a portion of the uninfected host cells with at least a portion of the virally infected cells; and
incubating the inoculated cells in the presence of incubation media for a time and under conditions sufficient to provide a composition in which at least 20% of the cells in the composition are virally infected.

57. The method according to claim 56, wherein the number of virally infected cells added to the uninfected host cells represent no more than 10% of the total number of cells.

58. The method according to claim 56, wherein the number of virally infected cells added to the uninfected host cells represent no more than 1% of the total number of cells.

59. The method according to claim 56, wherein the viability of cells contained in the cryogenic vessel is at least 70%.

60. The method according to claim 56, wherein the viability of cells contained in the cryogenic vessel is at least 90%.

61. The method according to claim 56, wherein the admixture of virally infected cells and cryo-protective agent contained in the cryogenic vessel is at a temperature of less than or equal to −70° C.

62. The method according to claim 56, wherein the inoculated cells are incubated for a time and under conditions sufficient so that at least 50% of the cells in the composition are virally infected.

63. The method according to claim 56, wherein the inoculated cells are incubated for a time and under conditions sufficient so that the concentration of viruses in the composition is from 10⁷ viruses/ml to 10¹¹ viruses/ml.

64. The method according to claim 56, wherein the inoculated cells are incubated for a time and under conditions sufficient so that the concentration of polypeptide in the composition is from 0.0001 mg/ml to 10 mg/ml.

65. The method according to claim 56, wherein the virally infected cells are eukaryotic cells.

66. The method according to claim 56, wherein the virally infected cells are human cells.

67. The method according to claim 56, wherein the virally infected cells are insect cells.

68. The method according to claim 56, wherein the uninfected host cells are eukaryotic cells.

69. The method according to claim 56, wherein the uninfected host cells are human cells.

70. The method according to claim 56, wherein the uninfected host cells are insect cells.

71. The method according to claim 56, wherein the virally infected cells are infected with recombinant virus.

72. The method according to claim 56, further comprising isolating viral products contained in and/or released from the incubated cells.

73. The method according to claim 72, further comprising lysing the incubated cells to release viral products prior to isolating viral products.

74. The method according to claim 72, wherein the viral products comprise a recombinant polypeptide.

75. The method according to claim 72, wherein the viral products comprise virus.

76. A method for selecting virally infected cells comprising:
inoculating a plurality of uninfected host cells with a plurality of viruses;
incubating the inoculated cells in the presence of incubation media for a time and under conditions sufficient to obtain a plurality of virally infected cells and monitoring the cell viability and average cell diameter of the incubated cells during the incubation; and
stopping the incubation at a point in time when the incubated cells have a viability of at least 50% and an average cell diameter that has increased by at least 0.5 μm.

77. The method for selecting virally infected cells according to claim 76, wherein the incubation is stopped at a point in time when the incubated cells have a viability of at least 90%.

78. The method for selecting virally infected cells according to claim 76, wherein the incubation is stopped at a point in time when the incubated cells have an average cell diameter that has increased by at least 1 μm.

79. The method for selecting virally infected cells according to claim 76, wherein the incubation is stopped at a point in time when the incubated cells have a viability of at least 90% and an average cell diameter that has increased by at least 1 μm.

80. The method for selecting virally infected cells according to claim 76, wherein the monitoring comprises removing at least one aliquot of the incubating cells and determining the cell viability and average cell diameter of the cells in the aliquot in less than 10 minutes following removal of the aliquot.

81. The method for selecting virally infected cells according to claim 80, wherein determining the cell viability and average cell diameter of the cells in the aliquot comprises placing at least a portion of the cells in an automated apparatus capable of measuring cell viability and average cell diameter of cells, and observing and/or recording the measured cell viability and the average cell diameter of the cells.

82. The method for selecting virally infected cells according to claim 76, wherein the incubation is stopped by lowering the temperature of the incubated cells to 10° C. or lower.

83. The method for selecting virally infected cells according to claim 76, wherein the incubation is stopped by centrifuging the incubated cells and incubation media to provide a pellet of cells and then removing the supernatant.
